(19) United States
(12) Patent Application Publication
(10) Pub. No.: US 2006/0094010 A1
(43) Pub. Date: May 4, 2006

(54) Methods and compositions for analyzing comprised sample using single nucleotide polymorphism panels

(76) Inventors: Robert C. Giles, Farmer's Branch, TX (US); Jeanine M. Baisch, Dallas, TX (US); Brian McKeown, Headington (GB)

Correspondence Address: KALOW & SPRINGUT LLP, 488 MADISON AVENUE, 19TH FLOOR, NEW YORK, NY, 10022, US

(21) Appl. No.: 10/519,224

(22) PCT Filed: Jun. 26, 2003
(86) PCT No.: PCT/US03/20150

§ 371 Date: Oct. 14, 2005

Related U.S. Application Data
(60) Provisional application No. 60/392,504, filed on Jun. 28, 2002.

(51) Int. Cl. C12Q 1/68

(52) U.S. Cl. 435/6

(57) ABSTRACT

The present invention provides methods and compositions for analyzing compromised nucleic acid samples. The present invention also includes methods of selecting panels and panels of single nucleotide polymorphisms that are selected so as to be outside of tandem repeat regions, and are not genetically linked.

### FIELD OF THE INVENTION

The invention relates to methods and compositions for analyzing compromised nucleic acid samples.

### BACKGROUND OF THE INVENTION

Classical genetics, the discovery that genes are defined by nucleic acid sequences, the discovery of the structure of hereditary material, and the biotechnology revolution have given rise to the science of human identification by nucleic acid analysis. Great strides have been made toward systems capable of identifying the source of a sample of nucleic acids with a high degree of confidence from intact samples of genetic material.

A wide variety of nucleic acid analysis techniques are available for applications aimed at revealing genetic similarities between samples of nucleic acids. For example, highly polymorphic repetitive sequences that exist in genomes may be employed in genetic identification applications. These applications allow for identification of individuals in a population with a high degree of confidence. One important application relies upon the analysis of polymorphic tandem repeat loci. One example of a genetic identification application is the FBI's Combined DNA Index System, or CODIS, which employs thirteen polymorphic short tandem repeat loci for genetic identification.

Tandem repeat loci are loci in a genome that contain repeat units of nucleotide sequences of varying length, such as dinucleotide repeats, trinucleotide repeats, tetranucleotide repeats, and so forth. The length of the repeating unit varies from as small as two nucleotides to extremely large numbers of nucleotides. The repeats may be simple tandem sequence repeats or complex combinations thereof. Variations in the length or character of these repeats at such loci are referred to as polymorphisms at these loci. Such polymorphisms most frequently arise through the existence of varying numbers of such repeats at a locus between individuals in a population. By some estimates, tandem repeats are encountered in the human genome at an average frequency of about 15 kilobases. The number of alleles, or varieties of sequence repeats at a locus, typically vary from about as few as three or four to as many as fifteen or up to fifty or more. Their relative high frequency of occurrence, coupled with their significant degree of polymorphism, render these features of the genome attractive candidates for genetic identification applications. By examining a sufficient number of polymorphic tandem repeat loci in a non-compromised sample of nucleic acids and comparing the characteristics of the loci of that individual with the characteristics of the same loci in a reference sample from a second individual, a determination can be made as to whether the individual is genetically related to the second individual from whom the reference sample was obtained. Generally, polymorphic repeat loci employed in genetic identification applications are selected so as to be unlinked, or in Hardy-Weinberg equilibrium, with one another.

Various types of tandem repeat loci are employed in genetic identification applications. Short tandem repeats (STRs) arise from variations in the number of short stretches of nucleic acid sequences. In the human genome, STRs are believed to occur about once in every few hundred thousand bases. STRs span about 2-7 bases, and vary with respect to the number of repeat units they contain and exist as both simple and complex repeats. Another type of tandem repeat, minisatellite repeats, are usually about 10 to 50 or so bases repeated about 20-50 times. Microsatellite repeats are typically about 1-6 bases repeated up to six or more times. These repeats may occur many thousands of times throughout the genome. The nomenclature for tandem repeat loci is inexact. These and other tandem repeats may be referred to by the general, all-encompassing term variable numbers of tandem repeats, or VNTRs.

Genetic identification applications employing VNTRs can employ restriction fragment length polymorphism analysis (RFLP analysis), a gel-based method, or methods based on the polymerase chain reaction (PCR). RFLP analysis capitalizes on the differences in length between fragments of nucleic acids generated from non-compromised samples of nucleic acids by the use of restriction endonucleases. Restriction endonucleases, endonucleases for short, are enzymes that fragment, or cut, nucleic acids at highly predictable positions. If two intact samples of nucleic acids are cut by the same endonuclease, their fragment pattern will be identical if their genetic sequence is identical. If the samples are different, they will generate different fragments, based in part on the selection of cut sites at positions that will yield predictably different fragment sizes depending upon the occurrence of polymorphic tandem repeat loci within or at the cut site of a predicted fragment. Like many genetic identification applications employing tandem repeat loci, RFLP analysis relies upon the ability to separate, or resolve, the nucleic acid fragments based on their electrophoretic mobility through a sizing gel, or on other sizing protocols. Sizing-based protocols, however, are inherently limited by the resolving power of the sizing method; fragments that are either too small or differ only very slightly in size may not be resolvable. Although potentially a powerful genetic identification application, RFLP analysis generally requires fairly intact nucleic acid samples. Further, RFLP analysis requires considerable amounts of nucleic acids and requires a relatively long amount of time to generate and interpret results.

Genetic identification applications employing tandem repeat loci and PCR require less nucleic acids. In PCR-based applications, sequences containing loci with tandem repeat sequences are amplified, or copied, many times over and then typically separated and identified using sizing protocols. However, due to the nature of the PCR polymerase, and the nature of tandem repeat loci, PCR methods are prone to artifactual results due to “slippage,” or “stutter” during PCR amplification. Such slippage or stutter is due to the inability of the polymerizing enzyme to faithfully and accurately copy the sequences containing the tandem repeats. The nature of the tandem repeat sequence causes the PCR polymerase to sometimes skip and sometimes over-copy elements of the repeating units. As a result, the amplified copy of the sequence containing the tandem repeat is either longer or shorter than the original, thus failing to provide the fidelity required for genetic identification applications. Further, most PCR-based applications rely upon sizing methods for identification, and thus have the same drawbacks in this respect as does RFLP analysis. Due to the length of many useful tandem repeat loci, the amplified or copied sequences must be generally at least near a hundred and up to a thousand or more bases in length. Compromised nucleic acid samples may not be so intact as to contain a sufficient number of tandem repeat loci useful in genetic identification applications.

Employment of existing genetic identification applications is often precluded due to the compromised nature of the sample containing the nucleic acids of uncertain identity or origin. Many factors may contribute to the inability to extract genetic information from a compromised sample. The sample may have been exposed to physical forces, such as heat or shear forces, ultraviolet light from, for example, the sun. The sample may have been subjected to a plethora of chemical degradative agents, and a wide variety of biological degradative processes, such as, for example, exposure to microorganisms or nucleases. These processes may result in a sample that comprises fewer than the optimal number of intact useful loci available for genetic analysis, rendering the compromised sample uninformative to currently available genetic identification applications.

Thus, there is a need for genetic identification applications for use with compromised nucleic acid samples that do not necessarily rely on sizing protocols for identification, and do not rely on the existence of sufficient tandem repeat loci for identification purposes.

### SUMMARY OF THE INVENTION

In one embodiment, the invention comprises a panel of single nucleotide polymorphisms useful for determining human identity from a compromised sample. In another embodiment of the invention, the single nucleotide polymorphisms of the panel include the nucleic acid sequences selected from the group consisting of SEQ ID NOS. 25-36, 61-72, 98-109, 134-145, 170-181, 206-217, 242-253, 278-289, 314-325, 351-362, 387-398, 423-434, and 457-467.

In yet another embodiment, the invention comprises a method of generating a panel of single nucleotide polymorphisms from a population of interest for analyzing a compromised nucleic acid sample, comprising: selecting a panel of two or more single nucleotide polymorphisms in a genome of the population of interest, wherein each of the two or more single nucleotide polymorphisms of the panel are single nucleotide polymorphisms of the genome that are not genetically linked with respect to one another, and wherein each of the two or more single nucleotide polymorphisms of the panel are single nucleotide polymorphisms of the genome that are located outside tandem repeat nucleic acid sequences, thereby generating the panel of single nucleotide polymorphisms from the population of interest for analyzing the compromised nucleic acid sample. In another embodiment, the invention comprises a method wherein the compromised sample comprises nucleic acids from about 10 nucleotides in length to about 100 nucleotides in length. In another embodiment, a method is employed wherein the population of interest is human. Yet another embodiment of the invention employs a method wherein the population of interest is one missing human.

In another embodiment, the invention comprises a method for determining the identity of an individual from an unknown sample of compromised nucleic acids, comprising: obtaining the unknown sample of compromised nucleic acids having two or more single nucleotide polymorphisms from an individual; identifying two or more single nucleotide polymorphisms present in the unknown sample of compromised nucleic acids; comparing the identity of each of the two or more single nucleotides polymorphisms in the compromised sample with a panel of single nucleotide polymorphisms from a known sample to determine a number of matches between each of the two or more single nucleotide polymorphisms in the unknown sample and the panel, wherein the panel comprises two or more single nucleotide polymorphisms that are not genetically linked with respect to one another, and are located outside tandem repeat nucleic acid sequences; and determining the probability that the unknown sample and the known sample are derived from the same or related individual based on the number of matches between each of the two or more single nucleotide polymorphism in the unknown sample and the known sample, thereby determining the identity of the individual from the unknown sample of compromised nucleic acids.

Yet another embodiment of the invention comprises a method for determining the identity of an individual from an unknown sample of compromised nucleic acids, comprising: obtaining the unknown sample of compromised nucleic acids having two or more single nucleotide polymorphisms from an individual; obtaining a known sample of nucleic acids having two or more single nucleotide polymorphisms; selecting a panel of two or more single nucleotide polymorphisms, wherein each of the two or more single nucleotide polymorphisms of the panel are not genetically linked with respect to one another, and wherein each of the single nucleotide polymorphisms of the panel are located outside tandem repeat nucleic acid sequences; determining the identity of each of the two or more single nucleotide polymorphisms of the panel that are present in the compromised nucleic acid sample; determining the identity of each of the two or more single nucleotide polymorphisms of the panel that are present in the known sample; comparing the identities of the two or more single nucleotide polymorphisms of the panel observed in the known sample with the identities of the two or more single nucleotide polymorphisms of the panel observed in the unknown sample of compromised nucleic acids; and determining the probability that the unknown sample and the known sample are derived from the same or related individual, thereby determining the identity of the individual from the unknown sample of compromised nucleic acids.

In another embodiment of the invention, the known sample and the unknown sample are from the same individual. Yet another embodiment of the invention comprises a method wherein the known sample is from a family member. In yet another embodiment, the compromised nucleic acid sample comprises nucleic acid fragments from about 10 nucleotides in length to about 100 nucleotides in length. In another embodiment, the identity of the one or more single nucleotide polymorphisms is determined using a single base primer extension reaction. In another embodiment, the two or more of the single nucleotide polymorphisms of the compromised sample are identified in a multiplexed reaction. In another embodiment, the two or more of the single nucleotide polymorphisms of the panel are identified in a multiplexed reaction. In another embodiment, the two or more single nucleotide polymorphisms of the panel are identified on an array. In another embodiment, the two or more single nucleotide polymorphisms of the compromised sample are identified on an array. In another embodiment, the array is an addressable array. In another embodiment, the array is an addressable array. In another embodiment, the array is a virtual array. In another embodiment, the array is a virtual array.

In yet another embodiment, the invention comprises a method for genotyping a compromised nucleic acid sample, comprising: obtaining the sample of compromised nucleic acids from an individual; identifying two or more single nucleotide polymorphisms present in the compromised nucleic acid sample; and comparing the identity of each of the two or more single nucleotides polymorphisms in the compromised sample with a panel of single nucleotide polymorphisms from a population of interest to determine the frequency of occurrence of each of the two or more single nucleotide polymorphism in the compromised sample with the population of interest, wherein the panel comprises two or more single nucleotide polymorphisms that are not genetically linked with respect to one another, and are located outside tandem repeat nucleic acid sequences; thereby genotyping the sample of compromised nucleic acids.

In still another embodiment, the invention comprises method for genotyping a compromised nucleic acid sample, comprising: obtaining the sample of compromised nucleic acids from an individual; selecting a panel of single nucleotide polymorphisms from a genome of a population of interest, the panel comprising two or more single nucleotide polymorphisms, wherein each of the two or more single nucleotide polymorphisms of the panel are single nucleotide polymorphisms that are not genetically linked with respect to one another and are located outside tandem repeat nucleic acid sequences; identifying two or more single nucleotide polymorphisms present in the compromised nucleic acid sample; and comparing the identities of the two or more single nucleotide polymorphisms observed in the compromised sample with the identities of the two or more single nucleotide polymorphisms observed in the panel to determine a genotype, thereby obtaining the genotype for the compromised nucleic acid sample. A further embodiment comprises a genotyping method wherein the single nucleotide polymorphisms of the panel are biallelic, and wherein the identity of the polymorphism in each allele is a T and/or C. In another embodiment, the invention includes a genotyping method wherein the population of interest is human. A further embodiment includes a genotyping method wherein the sample comprises human nucleic acids. Another embodiment comprises a genotyping method wherein the two or more single nucleotide polymorphisms present in the compromised nucleic acid sample are identified using a single base primer extension reaction. Yet another embodiment comprises a genotyping method wherein the two or more single nucleotide polymorphisms present in the compromised nucleic acid sample are identified in a multiplexed reaction. Another embodiment comprises a genotyping method wherein the two or more single nucleotide polymorphisms present in the compromised nucleic acid sample are identified on an array. A further embodiment comprises a genotyping method wherein the array is an addressable array. Still another embodiment comprises a genotyping method wherein the array is a virtual array. Yet another embodiment comprises a genotyping method wherein the compromised nucleic acid sample is amplified to a length of from about 10 nucleotides to about 100 nucleotides.

For a better understanding of the present invention together with other and further advantages and embodiments, reference is made to the following description taken in conjunction with the examples, the scope of which is set forth in the appended claims.

### BRIEF DESCRIPTION OF THE DRAWINGS

FIG. 1 depicts an embodiment of the invention wherein a compromised sample of nucleic acids is obtained; nucleic acids containing single nucleotide polymorphisms, or SNPs, are amplified employing the nucleic acids of the compromised sample as templates; the amplified nucleic acids containing single nucleotide polymorphisms are subjected to a primer extension reaction in which the primers are extended by a single base, for example, a labeled nucleotide derivative; the identity of the single nucleotide polymorphisms of the amplified nucleic acids are determined; the identity of each single nucleotide polymorphism determined from the amplified nucleic acids is compared with the identity of each corresponding single nucleotide polymorphism in a reference sample; and the likelihood that the nucleic acids of the compromised sample are genetically similar to the nucleic acids of the reference sample is determined.

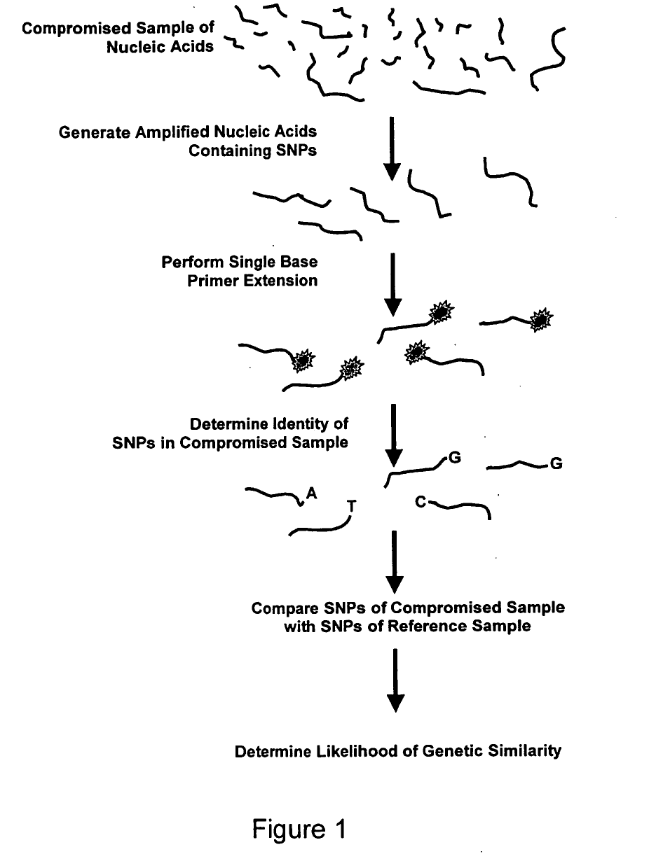

### DETAILED DESCRIPTION OF THE PREFERRED EMBODIMENTS

The present invention will now be described in connection with preferred embodiments. These embodiments are presented to aid in an understanding of the present invention and are not intended, and should not be construed, to limit the invention in any way. All alternatives, modifications and equivalents that may become obvious to those of ordinary skill upon reading the disclosure are included within the spirit and scope of the present invention.

This disclosure is not a primer on the analysis of compromised nucleic acids; basic concepts known to those skilled in the art or readily determinable have not been set forth in detail.

In one embodiment, the invention comprises a panel of single nucleotide polymorphisms for analyzing compromised nucleic acid samples, comprising two or more single nucleotide polymorphisms, wherein each of the two or more single nucleotide polymorphisms of the panel are selected from single nucleotide polymorphisms that are not genetically linked with respect to one another, and wherein each of the two or more single nucleotide polymorphisms of the panel are selected from single nucleotide polymorphisms that are located outside tandem repeat nucleic acid sequences.

By “panel” is meant a pre-selected group of single nucleotide polymorphisms suitable for use in identifying a member of a population. For example, in a preferred embodiment, the panel comprises a number of single nucleotide polymorphisms pre-selected from the single nucleotide polymorphisms of the human genome, wherein the single nucleotide polymorphisms are sufficient in number and character to genetically identify an individual to a degree of statistical certainty. Genetically identify includes the ability to distinguish one individual from another in a population by viewing the identity of the single nucleotide polymorphisms of the panel. The distinction of one individual from another is achieved, for example, by comparing the identities of the single nucleotide polymorphisms in the panel to a compromised sample containing all or some of the single nucleotide polymorphisms of the panel. Genetically identifying includes the establishment, to a degree of statistical certainty, of whether the single nucleotide polymorphisms in a compromised sample are the same or different from single nucleotide polymorphisms in a reference sample. The reference sample may, for example, comprise nucleic acids from another individual, such as a family member. By “genetically identify” is also meant the establishment, to a degree of statistical certainty, whether the single nucleotides in a compromised sample are the same or different from the single nucleotide polymorphisms of more than one reference sample. For example, the single nucleotide polymorphisms of a compromised sample can be compared to the single nucleotide polymorphisms in a group of reference samples, such as putative family members, to determine whether the nucleic acids of the compromised sample are derived from an individual or individuals genetically related to the individuals from which the one or more reference samples are derived.

“Comparing” single nucleotide polymorphisms means determining whether single nucleotide polymorphisms of one sample are identical or different from single nucleotide polymorphisms of a second sample, wherein one or both samples are compromised samples, or one sample is a compromised sample and one sample is a reference sample.

The reference sample may comprise single nucleotide polymorphisms determined from biological material taken from one or more donor individuals and wherein the identities of the single nucleotide polymorphisms are determined from the biological material. The reference sample may be any collection of single nucleotide polymorphisms whose identity is determined in any manner. For example, a reference sample may be a collection of identities of single nucleotide polymorphisms established without determining their existence through directly determining their identity from a biological sample of nucleic acids, but instead are generated by deducing nucleotide sequences from proteins, for example, or generating single nucleotide polymorphisms by observing single nucleotide polymorphisms in a group of family members. One reference sample, for example, would comprise the expected genotype of a member of a family, where the expected genotype of the family member is generated by observing the genotypes of other family members and, employing genetic algorithms and theories well known in the art, arriving at an expected genotype of the family member. In relation to the embodiments of the present invention, such an expected genotype would comprise a group of identities of single nucleotide polymorphisms the family member would be expected to display, as deduced from the genotypes of family members and through the use of genetic algorithms and theories known in the art.

Identifying an individual to “a degree of statistical certainty” is meant the establishment of a degree of statistical confidence that the compromised sample is related genetically to a reference sample or to another compromised sample. Many methods are known in the art of genetic identification to achieve this end. The algorithms and methods employed to arrive at statistical certainty in a given case may vary. For example, where the single nucleotide polymorphisms of a panel are identical between two samples or a sample and a reference sample, the degree of statistical certainty may be calculated from the individual probabilities that are associated with each allele in the samples or at each locus.

A compromised sample is “genetically related” to another compromised sample or a reference sample if the samples can be said, to a degree of statistical certainty, to derive from a defined population of interest. By a “defined population of interest” is meant a group of individuals of interest that share certain features of their genomes in common, for example, family members, ethnic groups such as Asians, Africans, Native Americans, and the like. A “defined population of interest” may be as small as a single individual, or as large a group as all females or all males in the human population. Thus, for example, a compromised sample derived from a male individual of Asian heritage may be “genetically related” to a female Asian sibling if the defined population of interest consists of all Asians, but would not be considered to be “genetically related” in this sense if the defined population of interest consists of Asian males only.

By “compromised nucleic acid sample” is meant a biological sample known to contain or suspected to contain nucleic acids, wherein the nucleic acids of the sample are too degraded. For example, genetic analysis of nucleic acid samples employing tandem repeat loci analysis, such as employed with identification systems relying on CODIS loci, cannot be reliably accomplished with nucleic acid samples that consist of fragments that do not contain a sufficient number of intact, forensically useful tandem repeat sequences. In reality, nucleic acid samples, particularly those employed for forensic analysis, may be significantly degraded. The sample may have been exposed to physical forces, such as heat or shear forces, ultraviolet light from, for example, the sun. The sample may have been subjected to a plethora of chemical degradative processes. The sample may have been subjected to a wide variety of biological degradative processes, such as, for example, exposure to microorganisms or nucleases. These processes may result in a sample that comprises fewer than the optimal number of intact useful loci available for genetic analysis employing methods known in the art that do not exploit single nucleotide polymorphisms, rendering the compromised sample uninformative to currently employed genetic identification applications. In a preferred embodiment of the invention, the compromised nucleic acid sample comprises nucleic acid fragments from about 10 nucleotides in length to about 100 nucleotides in length. Most preferably, the compromised nucleic acid is substantially comprised of nucleic acid fragments from at least 50 to at least about 100 nucleotides in length. In practice, the compromised sample may even comprise nucleic acid fragments that are as short as one or two nucleotides in length, as long as sufficient nucleic acids of length 10 to 100 nucleotides exist in the sample that bear enough single nucleotide polymorphisms to genotype the sample or identify an individual to a degree of statistical certainty. Likewise, the compromised sample may contain nucleotide fragments in excess of 100 nucleotides in length.

By “not genetically linked with respect to one another” is meant that the single nucleotide polymorphisms of the present invention are selected so as to be a desirable distance apart from one another if they reside on the same chromosome or nucleic acid molecule. Preferably, the single nucleotide polymorphisms of the panel are selected so as to be about ten to fifteen megabases apart. Most preferably, the single nucleotide polymorphisms of a panel are about 20 to about 100 or more megabases apart. Suitable single nucleotide polymorphisms include those that are not in linkage disequilibrium with respect to one another, although there is no need for any single nucleotide polymorphisms of any panel to be in perfect equilibrium. Suitable single nucleotide polymorphisms of a panel include those that are inherited independently of one another. That is to say, suitable single nucleotide polymorphisms may include those wherein no two single nucleotide polymorphisms of a panel are always inherited together.

Tandem repeat loci are loci in a genome that contain repeat units of nucleotide sequences of varying length, such as dinucleotide repeats, trinucleotide repeats, tetranucleotide repeats, and so forth. The length of the repeating unit varies from as small as two nucleotides to extremely large numbers of nucleotides. The repeats may be simple tandem sequence repeats or complex combinations thereof. Variations in the length or character of these repeats at such loci are referred to as polymorphisms at these loci. Such polymorphisms most frequently arise through the existence of varying numbers of such repeats at a locus between individuals in a population. By some estimates, tandem repeats are encountered in the human genome at an average frequency of about 15 kilobases. The number of alleles, or varieties of sequence repeats at a locus, typically vary from about as few as three or four to as many as fifteen or up to fifty or more. Their relative high frequency of occurrence, coupled with their significant degree of polymorphism, render these features of the genome attractive candidates for genetic identification applications. By examining a sufficient number of polymorphic tandem repeat loci in a non-compromised sample of nucleic acids and comparing the characteristics of the loci of that individual with the characteristics of the same loci in a reference sample from a second individual, a determination can be made as to whether the individual is genetically related to the second individual from whom the reference sample was obtained. Generally, polymorphic repeat loci employed in genetic identification applications are selected so as to be unlinked, or in Hardy-Weinberg equilibrium, with one another.

Various types of tandem repeat loci are employed in genetic identification applications. Short tandem repeats (STRs) arise from variations in the number of short stretches of nucleic acid sequences. In the human genome, STRs are believed to occur about once in every few hundred thousand bases. STRs span about 2-7 bases, and vary with respect to the number of repeat units they contain and exist as both simple and complex repeats. Another type of tandem repeat, minisatellite repeats, are usually about 10 to 50 or so bases repeated about 20-50 times. Microsatellite repeats are typically about 1-6 bases repeated up to six or more times. These repeats may occur many thousands of times throughout the genome. The nomenclature for tandem repeat loci is inexact. These and other tandem repeats may be referred to by the general, all-encompassing term variable numbers of tandem repeats, or VNTRs.

Another embodiment of the invention comprises a method of generating a panel of single nucleotide polymorphisms from a population of interest for analyzing a compromised nucleic acid sample, comprising selecting a panel of two or more single nucleotide polymorphisms in a genome of the population of interest, wherein each of the two or more single nucleotide polymorphisms of the panel are single nucleotide polymorphisms of the genome that are not genetically linked with respect to one another, and wherein each of the two or more single nucleotide polymorphisms of the panel are single nucleotide polymorphisms of the genome that are located outside tandem repeat nucleic acid sequences, thereby generating the panel of single nucleotide polymorphisms from the population of interest for analyzing the compromised nucleic acid sample.

By “generating a panel of single nucleotide polymorphisms” is meant the process of selecting suitable single nucleotide polymorphisms from a genome of interest, wherein the single nucleotide polymorphisms are useful in genetic analysis or identification. Generating a panel comprises selecting single nucleotide polymorphisms that are located outside of tandem repeat regions and are not genetically linked within the meaning of this invention. The single nucleotide polymorphisms are then analyzed by any method known in the art so as to select primers capable of identifying the single nucleotide polymorphisms in multiplex reactions. This analysis typically involves, for example, selecting polymorphisms wherein the detection primers and amplification primers will the same or similar melting and annealing temperatures for purposes of amplification and single base extension reactions.

One or more panels may be employed to analyze a single sample comprising compromised nucleic acids. The single nucleotide polymorphisms of the present invention are selected so as to be a desirable distance apart from one another if they reside on the same chromosome or nucleic acid molecule. Preferably, the single nucleotide polymorphisms of the panel are selected so as to be about ten to fifteen megabases apart. Most preferably, the single nucleotide polymorphisms of a panel are about 20 to about 100 or more megabases apart. Suitable single nucleotide polymorphisms include those that are not in linkage disequilibrium with respect to one another, although there is no need for any single nucleotide polymorphisms of any panel to be in perfect equilibrium. Suitable single nucleotide polymorphisms of a panel include those that are inherited independently of one another. That is to say, suitable single nucleotide polymorphisms may include those wherein no two single nucleotide polymorphisms of a panel are always inherited together. Most preferably, the single nucleotide polymorphisms of a panel are biallelic. Most preferably, the identities of the alleles of the single nucleotide polymorphisms a panel are all T/C.

Another embodiment of the invention comprises a method for determining the identity of an individual from an unknown sample of compromised nucleic acids, comprising obtaining the unknown sample of compromised nucleic acids having two or more single nucleotide polymorphisms from an individual; identifying two or more single nucleotide polymorphisms present in the unknown sample of compromised nucleic acids; comparing the identity of each of the two or more single nucleotides polymorphisms in the compromised sample with a panel of single nucleotide polymorphisms from a known sample to determine a number of matches between each of the two or more single nucleotide polymorphisms in the unknown sample and the panel, wherein the panel comprises two or more single nucleotide polymorphisms that are not genetically linked with respect to one another, and are located outside tandem repeat nucleic acid sequences; and determining the probability that the unknown sample and the known sample are derived from the same or related individual based on the number of matches between each of the two or more single nucleotide polymorphism in the unknown sample and the known sample, thereby determining the identity of the individual from the unknown sample of compromised nucleic acids.

By “determining the identity of an individual” is meant determining a characteristic of interest of the individual. In a preferred embodiment, “determining the identity of an individual” is determining who the individual is to the exclusion of all other individuals in a population of interest, to a high degree of statistical certainty. In the most preferred embodiment, “determining the identity of an individual” comprises identifying a single individual from the entire human population with a high degree of statistical certainty. Most preferably, the degree of statistical certainty is one in one billion or higher. Such a degree of certainty is attainable with about thirty single nucleotide polymorphisms. However, the invention may be employed wherein the compromised sample is compared to a reference wherein “determining the identity of an individual” requires a substantially lesser degree of statistical certainty.

By “unknown sample” is meant a sample of material known or suspected to comprise compromised nucleic acids, wherein the identity of the individual or individuals from whom the compromised nucleic acids is derived is not known, or not known with a desired degree of statistical certainty.

By “comparing the identity” of a single nucleotide polymorphism in a compromised sample to a single nucleotide polymorphism in another compromised sample or in a reference sample is meant determining whether the nucleotide at a single nucleotide polymorphic site in one sample is identical to the nucleotide at the same single nucleotide polymorphic site in a second sample. This comparison is carried out for each single nucleotide polymorphism analyzed, and a determination is made with respect to each single nucleotide polymorphic site whether a “match” exists. By “match” is meant exact identity of nucleic acids at a single nucleotide polymorphic site in two or more samples. Two or more samples that bear the same nucleotide on the same strand at a given single polymorphic site are said to “match” with respect to that site.

By “determining the probability that the unknown sample and the known sample are derived from the same or related individual” is meant comparing the identities of the nucleotides present at the single polymorphic sites in the unknown sample and the known sample, and calculating the statistical likelihood that the matches observed would occur by chance. Methods and algorithms for calculating the statistical likelihood that a match would occur by chance are well known in the art, and rely on the probability of a particular nucleotide being present at a particular locus.

By “known sample” is meant a sample of material known to contain nucleic acids, compromised or not compromised, wherein the identity of the individual or individuals from whom the known sample is derived is known, or is known with a desired degree of statistical certainty.

Another embodiment of the invention comprises a method for determining the identity of an individual from an unknown sample of compromised nucleic acids, comprising obtaining the unknown sample of compromised nucleic acids having two or more single nucleotide polymorphisms from an individual; obtaining a known sample of nucleic acids having two or more single nucleotide polymorphisms; selecting a panel of two or more single nucleotide polymorphisms, wherein each of the two or more single nucleotide polymorphisms of the panel are not genetically linked with respect to one another, and wherein each of the single nucleotide polymorphisms of the panel are located outside tandem repeat nucleic acid sequences; determining the identity of each of the two or more single nucleotide polymorphisms of the panel that are present in the compromised nucleic acid sample; and determining the identity of each of the two or more single nucleotide polymorphisms of the panel that are present in the known sample; comparing the identities of the two or more single nucleotide polymorphisms of the panel observed in the known sample with the identities of the two or more single nucleotide polymorphisms of the panel observed in the unknown sample of compromised nucleic acids; and determining the probability that the unknown sample and the known sample are derived from the same or related individual, thereby determining the identity of the individual from the unknown sample of compromised nucleic acids.

By “the known sample and the unknown sample are from the same individual” is meant that the source of the samples are derived from biological matter belonging to the same individual. One individual may be said to be “a family member” with respect to another individual if the two individuals are related by consanguinity of any degree to one another. Most preferably, “a family member” is related by siblingship or parentage.

By “single base primer extension” is meant hybridizing an extension primer on a target nucleic acid immediately adjacent to a polymorphic site, and, under conditions sufficient to allow primer extension in the presence of a polymerizing agent, extending the primer. Most preferably, the primer is extended by a single labeled terminating nucleotide. One preferred method of detecting polymorphic sites employs enzyme-assisted primer extension. SNP-IT™ (disclosed by Goelet, P. et al., and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, it is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design an oligonucleotide primer that is complementary to a region immediately adjacent to, but not including, the variable nucleotide(s) in the polymorphic site of the target polynucleotide. The target polynucleotide is isolated from a biological sample and hybridized to the interrogating primer. Following isolation, the target polynucleotide may be amplified by any suitable means prior to hybridization to the interrogating primer. The primer is extended by a single labeled terminator nucleotide, such as a dideoxynucleotide, using a polymerase, often in the presence of one or more chain terminating nucleoside triphosphate precursors (or suitable analogs). A detectable signal is thereby produced. As used herein, immediately adjacent to the polymorphic site includes from about 1 to about 100 nucleotides, more preferably from about 1 to about 25 nucleotides in the 5′ direction of the polymorphic site, with respect to the directionality of the target nucleic acid. Most preferably, the primer is hybridized one nucleotide immediately adjacent to the polymorphic site in the 5′ direction with respect to the polymorphic site.

In some embodiments of SNP-IT™, the primer is bound to a solid support prior to the extension reaction. In other embodiments, the extension reaction is performed in solution (such as in a test tube or a microwell) and the extended product is subsequently bound to a solid support. In an alternate embodiment of SNP-IT™, the primer is detectably labeled and the extended terminator nucleotide is modified so as to enable the extended primer product to be bound to a solid support. An example of this includes where the primer is fluorescently labeled and the terminator nucleotide is a biotin-labeled terminator nucleotide and the solid support is coated or derivatized with avidin or streptavidin. In such embodiments, an extended primer would thus be capable of binding to a solid support and non-extended primers would be unable to bind to the support, thereby producing a detectable signal dependent upon a successful extension reaction.

Ligase/polymerase mediated genetic bit analysis (U.S. Pat. Nos. 5,679,524, and 5,952,174, both herein incorporated by reference) is another example of a suitable polymerase mediated primer extension method for determining the identity of a nucleotide at a polymorphic site. Ligase/polymerase SNP-IT™ utilizes two primers. Generally, one primer is detectably labeled, while the other is designed to be affixed to a solid support. In alternate embodiments of ligase/polymerase SNP-IT™, the extended nucleotide is detectably labeled. The primers in ligase/polymerase SNP-IT™ are designed to hybridize to each side of a polymorphic site, such that there is a gap comprising the polymorphic site. Only a successful extension reaction, followed by a successful ligation reaction, enables production of the detectable signal. The method offers the advantages of producing a signal with considerably lower background than is possible by methods employing either hybridization or primer extension alone.

An alternate method for determining the identity of a nucleotide at a polymorphic site in a target polynucleotide is described in Soderlund et al., U.S. Pat. No. 6,013,431 (the entire disclosure of which is herein incorporated by reference). In this method, the nucleotide sequence surrounding a polymorphic site in a target nucleic acid sequence is used to design an oligonucleotide primer that is complementary to a region flanking the 5′ end, with respect to the polymorphic site, of the target polynucleotide, but not including the variable nucleotide(s) in the polymorphic site of the target polynucleotide. The target polynucleotide is isolated from the biological sample and hybridized with an interrogating primer. In some embodiments of this method, following isolation, the target polynucleotide may be amplified by any suitable means prior to hybridization with the interrogating primer. The primer is extended, using a polymerase, often in the presence of a mixture of at least one labeled deoxynucleotide and one or more chain terminating nucleoside triphosphate precursors (or suitable analogs). A detectable signal is produced on the primer upon incorporation of the labeled deoxynucleotide into the primer.

The primer extension reaction of the present invention employs a mixture of one or more labeled nucleotides and a polymerizing agent. The term “nucleotide” or nucleic acid as used herein is intended to refer to ribonucleotides, deoxyribonucleotides, acyclic derivatives of nucleotides, and functional equivalents or derivatives thereof, of any phosphorylation state capable of being added to a primer by a polymerizing agent. Functional equivalents of nucleotides are those that act as substrates for a polymerase as, for example, in an amplification method or a primer extension method. Functional equivalents of nucleotides are also those that may be formed into a polynucleotide that retains the ability to hybridize in a sequence-specific manner to a target polynucleotide. Examples of nucleotides include chain-terminating nucleotides, most preferably dideoxynucleoside triphosphates (ddNTPs), such as ddATP, ddCTP, ddGTP, and ddTTP; however other terminators known to those skilled in the art, such as, for example, acyclo nucleotide analogs, other acyclo analogs, and arabinoside triphosphates, are also within the scope of the present invention. Preferred ddNTPs differ from conventional 2′deoxynucleoside triphosphates (dNTPs) in that they lack a hydroxyl group at the 3′position of the sugar component.

The nucleotides employed may bear a detectable characteristic. As used herein a detectable characteristic includes any identifiable characteristic that enables distinction between nucleotides. It is important that the detectable characteristic does not interfere with any of the methods of the present invention. Detectable characteristic refers to an atom or molecule or portion of a molecule that is capable of being detected employing an appropriate method of detection. Detectable characteristics include inherent mass, electric charge, electron spin, mass tag, radioactive isotope, dye, bioluminescence, chemiluminescence, nucleic acid characteristics, haptens, proteins, light scattering/phase shifting characteristics, or fluorescent characteristics.

Nucleotides and primers may be labeled according to any technique known in the art. Preferred labels include radiolabels, fluorescent labels, enzymatic labels, proteins, haptens, antibodies, sequence tags, mass tags, fluorescent tags and the like. Preferred dye type labels include, but are not limited to, TAMRA (carboxy-tetramethylrhodamine), ROX (carboxy-X-rhodamine), FAM (5-carboxyfluorescein), and the like.

The primer extension reaction of the present invention can employ one or more labeled nucleotide bases. Preferably, two or more nucleotides of different bases are employed. Most preferably, the primer extension reaction of the present invention employs four nucleotides of different bases. In the most preferred embodiment all four different types of nucleotide are labeled with distinguishable labels. For example, A labeled with dR6G, C labeled with dTAMRA, G labeled with dR110 and T labeled with dROX.

Once the primer extension reaction is employed, extended and unextended primers (if any) can be separated from each other so as to identify the polymorphic site on the one or more alleles that are interrogated. Separation of nucleic acids can be performed by any methods known in the art. Some separation methods include the detection of DNA duplexes with intercalating dyes such as, for example, ethidium bromide, hybridization methods to detect specific sequences and/or separate or capture oligonucleotide molecules whose structures are known or unknown and hybridization methods in connection with blotting methods well known in the art. Hybridization methods may be combined with other separation technologies well known in the art, such as separation of tagged oligonucleotides through solid phase capture, such as, for example, capture of hapten-linked oligonucleotides to immunoaffinity beads, which in turn may bear magnetic properties. Solid phase capture technologies also includes DNA affinity chromatography, wherein an oligonucleotide is captured by an immobilized oligonucleotide bearing a complementary sequence. Specific polynucleotide tags may be engineered into oligonucleotide primers, and separated by hybridization with immobilized complementary sequences. Such solid phase capture technologies also includes capture onto streptavidin-coated beads (magnetic or nonmagnetic) of biotinylated oligonucleotides. DNA may also be separated and with more traditional methods such as centrifugation, electrophoretic methods or precipitation or surface deposition methods. This is particularly so when the extended or unextended primers are in solution phase. The term “solution phase” is used herein to refer to a homogenous or heterogenous mixture. Such a mixture may be aqueous, organic, or contain both aqueous and organic components. As used herein, the term “solution” should be construed to be synonymous with suspension in that it should be construed to include particles suspended in a liquid medium.

The polymorphic sites can be detected by any means known in the art. One method of detection of nucleotides is by fluorescent techniques. Fluorescent hybridization probes may, for example, be constructed that are quenched in the absence of hybridization to target nucleic acid sequences. Other methods capitalize on energy transfer effects between fluorophores with overlapping absorption and emission spectra, such that signals are detected when two fluorophores are in close proximity to one another, as when captured or hybridized.

Nucleotides may also be detected by, or labeled with moieties that can be detected by, a variety of spectroscopic methods relating to the behavior of electromagnetic radiation. These spectroscopic methods include, for example, electron spin resonance, optical activity or rotation spectroscopy such as circular dichroism spectroscopy, fluorescence, fluorescence polarization, absorption/emission spectroscopy, ultraviolet, infrared, visible or mass spectroscopy, Raman spectroscopy and nuclear magnetic resonance spectroscopy.

Nucleotides and analogs thereof, terminators and/or primers may be labeled according to any technique known in the art. Preferred labels include radiolabels, fluorescent labels, enzymatic labels, proteins, haptens, antibodies, sequence tags, mass tags, fluorescent tags and the like. Preferred dye type labels include, but are not limited to, TAMRA (carboxy-tetramethylrhodamine), ROX (carboxy-X-rhodamine), FAM (5-carboxyfluorescein), and the like.

The term “detection” refers to identification of a detectable moiety or moieties. The term is intended to include the ability to identify a moiety by electromagnetic characteristics, such as, for example, charge, light, fluorescence, chemiluminescence, changes in electromagnetic characteristics such as, for example, fluorescence polarization, light polarization, dichroism, light scattering, changes in refractive index, reflection, infrared, ultraviolet, and visible spectra, mass, mass:charge ratio and all manner of detection technologies dependent upon electromagnetic radiation or changes in electromagnetic radiation. The term is also intended to include identification of a moiety based on binding affinity, intrinsic mass, mass deposition, and electrostatic properties, size and sequence length. It should be noted that characteristics such as mass and molecular weight may be estimated by apparent mass or apparent molecular weight, so the terms “mass” or “molecular weight” as used herein do not exclude estimations as determined by a variety of instrumentation and methods, and thus do not restrict these terms to any single absolute value without reference to the method or instrumentation used to arrive at the mass or molecular weight.

Another method of detecting the nucleotide present at the polymorphic site is by comparison of the concentrations of free, unincorporated nucleotides remaining in the reaction mixture at any point after the primer extension reaction. Mass spectroscopy in general and, for example, electrospray mass spectroscopy, may be employed for the detection of unincorporated nucleotides in this embodiment. This detection method is possible because only the nucleotide(s) complementary to the polymorphic base is (are) depleted in the reaction mixture during the primer extension reaction. Thus, mass spectrometry may be employed to compare the relative intensities of the mass peaks for the nucleotides. Likewise, the concentrations of unlabeled primers may be determined and the information employed to arrive at the identity of the nucleotide present at the polymorphic site.

Primers can be polynucleotides or oligonucleotides capable of being extended in a primer extension reaction at their 3′ end. As used herein, the term “polynucleotide” includes nucleotide polymers of any number. The term “oligonucleotide” includes a polynucleotide molecule comprising any number of nucleotides, preferably, less than about 100 nucleotides. More preferably, oligonucleotides are between 5 and 100 nucleotides in length. Most preferably, oligonucleotides are 15 to 60 nucleotides in length. The exact length of a particular oligonucleotide or polynucleotide, however, will depend on many factors, which in turn depend on its ultimate function or use. Some factors affecting the length of an oligonucleotide are, for example, the sequence of the oligonucleotide, the assay conditions in terms of such variables as salt concentrations and temperatures used during the assay, and whether or not the oligonucleotide is modified at the 5′ terminus to include additional bases for the purposes of modifying the mass:charge ratio of the oligonucleotide, and/or providing a tag capture sequence which may be used to geographically separate an oligonucleotide to a specific hybridization location on a DNA chip or array. Short primers may require lower temperatures to form sufficiently stable hybrid complexes with a template. The primers of the present invention should be complementary to the upper or lower strand target nucleic acids. Preferably, the initial amplification primers should not have self complementarity involving their 3‘ends’ in order to avoid primer fold back leading to self-priming architectures and assay noise. Preferred primers of the present invention include oligonucleotides from about 8 to about 40 nucleotides in length. Most preferably, the PCR primers are between 18 and 25 bases in length. Most preferably, SNP-IT™ primers (Orchid Biosciences, Inc.) are used as extension primers to determine the identity of the nucleotide at the polymorphic site. Most preferably, the SNP-IT™ primers are 40 to 45 base pairs in length, comprised of a 20 to 25 base pair 3′-region that is complementary to the sequence adjacent to the polymorphic locus, and a 20 base pair tag that is not complementary to any of the sample nucleic acid sequences.

Primers of about 10 nucleotides are the shortest sequence that can be used to selectively hybridize to a complementary target nucleic acid sequence against the background of non-target nucleic acids in the present state of the art. Most preferably, sequences of unbroken complementarity over at least 20 to about 35 nucleotides are used to assure a sufficient level of hybridization specificity, although length may vary considerably given the sequence of the target DNA molecule. The primers of this invention must be capable of specifically hybridizing to the target nucleic acid sequence—such as, for example, one or more upper primers hybridizing to one or more upper strand target nucleic acids or one or more lower strand nucleic acids. As used herein, two nucleic acid sequences are said to be capable of specifically hybridizing to one another if the two molecules are capable of forming an anti-parallel, double-stranded nucleic acid structure or hybrid under conditions sufficient to promote such hybridization, whereas they must be substantially unable to form a double-stranded structure or hybrid with one another when incubated with a non-target nucleic acid sequence under the same conditions.

A nucleic acid molecule is said to be the “complement” of another nucleic acid molecule—or itself—if it exhibits complete sequence complementarity. As used herein, molecules are said to exhibit “complete complementarity” when every nucleotide of one of the molecules is able to form a base pair with a nucleotide of the other. “Substantially complementary” refers to the ability to hybridize to one another—or with itself—with sufficient stability to permit annealing under at least under at least conventional low-stringency conditions. Similarly, the molecules are said to be “complementary” if they can hybridize to one another with sufficient stability to permit them to remain annealed to one another under conventional high-stringency conditions. Conventional stringency conditions are described, for example, in Sambrook, J., et al., Molecular Cloning, A Laboratory Manual, 2nd Edition, Cold Spring Harbor Press, Cold Spring Harbor, N.Y. (1989), herein incorporated by reference). Departures from complete complementarity are therefore permissible, as long as such departures do not completely preclude the capacity of the molecules to form a double-stranded structure or hybrid.

Primers employed in practicing the present invention may be tagged at the 5′ end. Tags include any label such as radioactive labels, fluorescent labels, enzymatic labels, proteins, haptens, antibodies, sequence tags, and the like. Preferably, the tag does not interfere with the processes of the present invention. Typically, a tag may be attached to the 5′ end of the primer, with the remainder of the primer sequence being complementary to the target nucleic acid. A preferred tag includes unique tags or marking each type of primer with a distinct sequence that is complementary to a sequence bound to a solid support, where such solid support may include an array, including an addressable array. Thus, when the primer is exposed to the solid support under suitable hybridization conditions, the tag hybridizes with the complementary sequence bound to the solid support. In this way, the identity of the primer can be determined by geometric location on the array, or by other means of identifying the point of association of the tag with the probe. Sequences complementary to the 5′ tag can be bound to a solid support at discrete positions on, for example, an addressable array.

Polymerizing agents useful in the present invention may be isolated or cloned from a variety of organisms including viruses, bacteria, archaebacteria, fungi, mycoplasma, prokaryotes, and eukaryotes. Preferred polymerizing agents include polymerases. Preferred polymerases for performing single base extensions using the methods and apparatus of the invention are polymerases exhibiting little or no exonuclease activity. More preferred are polymerases that tolerate and are active at temperatures greater than physiological temperatures, for example, at 50° C. to 70° C. or are tolerant of temperatures of at least 90° C. to about 95° C. Preferred polymerases include Taq® polymerase from T. aquaticus (commercially available from ABI, Foster City, Calif.), Sequenase® and ThermoSequenase® (commercially available from U.S. Biochemical, Cleveland, Ohio), and Exo(−) polymerase (commercially available from New England Biolabs, Beverley, Mass.) and AmpliTaq Gold®. Any polymerases exhibiting thermal stability may also be employed, such as for example, polymerases from Thermus species, including Thermus aquaticus, Thermus brocianus, Thermus thermophilus, and Thermus flavus; Pyrococcus species, including Pyrococcus furiosus, Pyrococcus sp. GB-D, and Pyrococcus woesei, Thermococcus litoralis, and Thermogata maritime. Biologically active proteolytic fragments, recombinant polymerases, genetically engineered polymerizing enzymes, and modified polymerases are included in the definition of polymerizing agent. It should be understood that the invention can employ various types of polymerases from various species and origins without undue experimentation.

By “multiplexed reaction” is meant the identification of two or more single nucleotide polymorphisms in a single reaction. A “multiplexed reaction” also includes the preparation, for example by amplification, of two or more target nucleic acids present in a compromised sample, coupled with the identification of two or more single nucleotide polymorphisms in a single reaction. Preferably, in a “multiplexed reaction” between at least about 10 to about 50 single nucleotide polymorphisms are identified in a single reaction. Most preferably, about 12 target nucleic acids are prepared, for example by amplification, and about to about 12 single nucleotide polymorphisms are identified in a single reaction. Preferably, primers employed to amplify the nucleic acids from the compromised sample exhibit similar melting temperatures, such that multiple amplicons comprising single nucleotide polymorphisms of one or more panels can be generated in a single reaction. Most preferably, about 12 amplicons are generated in a single reaction. Selection of single nucleotide polymorphisms of a panel for multiplexing purposes may be achieved by any method known in the art that can select extension primers based upon similarity of melting temperatures. Most preferably, nucleic acid sequences comprising single nucleotide polymorphisms that are about 20 to 100 megabases apart, and are biallelic T/C polymorphisms that are biallelic, are selected and inputted into Autoprimer software (http://www.autoprimer.com, herein incorporated by reference), and Autoprimer provides panels of about 12 single nucleotide polymorphisms that are suitable for use in multiplexed amplification and single base extension reactions based on melting temperature of the primers.

The extended primers can be separated and identified by any method known in the art. A preferable method of separating and identifying primer extension products is by capillary gel electrophoreses wherein a fluorescence detector is employed to identify primer extension products labeled with fluorescent terminating nucleotides. In this embodiment, extended primers bearing fluorescent labels are separated by their mass:charge ratio. Most preferably, SNP-IT™ primers (Orchid Biosciences, Inc.) are employed that bear tag capture sequences at their 5′-ends. In this embodiment, following single base primer extension at the SNP site with a fluorescent terminator, the reaction mixture is applied to an array bearing sequences complementary to the tag capture sequences of the primers, wherein the placement of the position of such complementary sequences on the array are known. In this embodiment, an appropriate fluorescent signal at a known position on an array indicates the identity of the nucleotide present at the SNP site. Most preferably, the assays are carried out using a SNPstream UHT Assay Kit™ (Orchid Biosciences, Inc.) and the identification is achieved using a SNPstream UHT Array Imager™ with a SNPstream Laser Enclosure™ coupled to a Control Computer, Data Analysis Computer, Server Computer and a SNPStream Data Analysis Software Suite™ (all from Orchid Biosciences, Inc.). However, many separation and detection methods are known to those skilled in the art, and the invention herein is amenable to a wide variety of detection and separation protocols.

Preferred separation methods employ exposing any extended and unextended primers to a solid support. Solid supports include arrays. The term “array” is used herein to refer to an ordered arrangement of immobilized biological molecules at a plurality of positions on a solid, semi-solid, gel or polymer phase. This definition includes phases treated or coated with silica, silane, silicon, silicates and derivatives thereof, plastics and derivatives thereof such as, for example, polystyrene, nylon and, in particular, polystyrene plates, glasses and derivatives thereof, including derivatized glass, glass beads, controlled pore glass (CPG). Immobilized biological molecules includes oligonucleotides that may include other moieties, such as tags and/or affinity moieties. The term “array” is intended to include and be synonymous with the terms “chip,” “biochip,” “biochip array,” “DNA chip,” “RNA chip,” “nucleotide chip,” and “oligonucleotide chip.” All these terms are intended to include arrays of arrays, and are intended to include arrays of biological polymers such as, for example, oligonucleotides and DNA molecules whose sequences are known or whose sequences are not known

Preferred arrays for the present invention include, but are not limited to, addressable arrays including an array as defined above wherein individual positions have known coordinates such that a signal at a given position on an array may be identified as having a particular identifiable characteristic. The terms “chip,” “biochip,” “biochip array,” “DNA chip,” “RNA chip,” “nucleotide chip,” and “oligonucleotide chip,” are intended to include combinations of arrays and microarrays. These terms are also intended to include arrays in any shape or configuration, 2-dimensional arrays, and 3-dimensional arrays.

A preferred array is the GenFlex™ Tag Array, from Affymetrix, Inc., that is comprised of capture probes for 2000 tag sequences. These are 20mers selected from all possible 20mers to have similar hybridization characteristics and at least minimal homology to sequences in the public databases. The most preferred array is the SNPstream UHT Array™ (Orchid Biosciences, Inc.).

Another preferred array is the addressable array that has sequence tags that complement any 5′ tags of primers employed in the present invention. These complementary tags are bound to the array at known positions. This type of tag hybridizes with the array under suitable hybridization conditions. By locating the bound primer in conjunction with detecting one or more extended primers, the nucleotide identity at the polymorphic site can be determined.

In one preferred embodiment of the present invention, the target nucleic acid sequences are arranged in a format that allows multiple simultaneous detections (multiplexing), as well as parallel processing using oligonucleotide arrays.

In another embodiment, the present invention includes virtual arrays where extended and unextended primers are separated on an array where the array comprises a suspension of microspheres, where the microspheres bear one or more capture moieties to separate the uniquely tagged primers. The microspheres, in turn, bear unique identifying characteristics such that they are capable of being separated on the basis of that characteristic, such as for example, diameter, density, size, color, and the like.

In another embodiment, the invention comprises a method for genotyping a compromised nucleic acid sample, comprising obtaining the sample of compromised nucleic acids from an individual; identifying two or more single nucleotide polymorphisms present in the compromised nucleic acid sample; and comparing the identity of each of the two or more single nucleotides polymorphisms in the compromised sample with a panel of single nucleotide polymorphisms from a population of interest to determine the frequency of occurrence of each of the two or more single nucleotide polymorphism in the compromised sample with the population of interest, wherein the panel comprises two or more single nucleotide polymorphisms that are not genetically linked with respect to one another, and are located outside tandem repeat nucleic acid sequences; thereby genotyping the sample of compromised nucleic acids.

By “genotyping” is meant first defining a set of genetic characteristics of interest, then determining the likelihood, to a degree of statistical certainty, whether the genetic characteristics of interest are present in a compromised nucleic acid sample. In one embodiment of the invention, the genetic characteristics of interest are a panel of single nucleotide polymorphisms in a population of interest, wherein the single nucleotide polymorphisms are not genetically linked with one another and are located outside tandem repeat nucleic acid sequences. A “genotype,” as used herein, is meant the identities of the nucleotides of the single nucleotide polymorphisms of the one or more panels that are found in a sample or a reference sample.

By “frequency of occurrence” of a single nucleotide polymorphism is meant the observed frequency that a particular nucleotide appears at a particular single nucleotide polymorphic site in a population of interest. Most preferably, the single nucleotide polymorphisms of the invention are biallelic, and the identity of the polymorphic nucleotides are T and/or C.

In another embodiment, the invention comprises a method for genotyping a compromised nucleic acid sample, comprising obtaining the sample of compromised nucleic acids from an individual; selecting a panel of single nucleotide polymorphisms from a genome of a population of interest, the panel comprising two or more single nucleotide polymorphisms, wherein each of the two or more single nucleotide polymorphisms of the panel are single nucleotide polymorphisms that are not genetically linked with respect to one another and are located outside tandem repeat nucleic acid sequences; identifying two or more single nucleotide polymorphisms present in the compromised nucleic acid sample; and comparing the identities of the two or more single nucleotide polymorphisms observed in the compromised sample with the identities of the two or more single nucleotide polymorphisms observed in the panel to determine a genotype, thereby obtaining the genotype for the compromised nucleic acid sample.

By “human nucleic acids” is meant any variety of nucleic acids derived from a human. “Human nucleic acids” is meant to include nucleic acid samples that comprise degraded or chemically or physically modified by the elements or otherwise, with the only limitation being that they are amenable to the identification or genotyping methods of the present invention.

By “amplified” is meant an increased number of target nucleic acids. In one embodiment of the invention, target nucleic acids of a compromised sample of nucleic acids are amplified by means of the polymerase chain reaction (PCR), employing PCR primers. “Amplified” is not meant to be limited to PCR, however. Amplification, as used herein, refers to any technique that increases quantities of target nucleic acids, including but not limited to hybridization or affinity methods for enriching the yield or number of target nucleic acids of interest.

By “target nucleic acids” is meant sequences of nucleic acids that contain one or more single nucleotide polymorphisms of interest. The target nucleic acid sequence will preferably be biologically active with regard to the capacity of this nucleic acid to hybridize to an oligonucleotide or a polynucleotide molecule. Target nucleic acid sequences may be either DNA or RNA, single-stranded or double-stranded or a DNA/RNA hybrid duplex. The target nucleic acid sequence may be a polynucleotide or oligonucleotide. Target nucleic acid sequences in the compromised nucleic acid samples of the invention are preferably about 10 to about 100 nucleotides in length. Most preferably, the target nucleic acid sequences in the compromised nucleic acid samples of the invention are about 10 to about 50 nucleotides in length. Methods of recovering degraded, compromised, and/or fractionated DNA are well known in the art, and include gel electrophoresis, HPLC and techniques which can capitalize, for example, on the recovery of various sequences on the basis of hybridization to a capture sequence.

The target nucleic acid may be isolated, or derived from a biological sample. The term “isolated” as used herein refers to the state of being substantially free of other material such as non nuclear proteins, lipids, carbohydrates, or other materials such as cellular debris or growth media with which the target nucleic acid may be associated. Typically, the term “isolated” is not intended to refer to a complete absence of these materials. Neither is the term “isolated” generally intended to refer to the absence of stabilizing agents such as water, buffers, or salts, unless they are present in amounts that substantially interfere with the methods of the present invention. The term “sample” as used herein generally refers to any material containing nucleic acid, either DNA or RNA or DNA/RNA hybrids. Samples can be from any source including plants and animals including humans. Generally, such material will be in the form of a blood sample, a tissue sample, cells directly from individuals or propagated in culture, plants, yeast, fungi, mycoplasma, viruses, archaebacteria, histology sections, or buccal swabs, either fresh, fixed, frozen, or embedded in paraffin or another fixative. Such a sample is amenable to template preparation by, for example, alkali lysis. Other sample types will be amenable to assay, but may require different or more extensive template preparation such as, for example, by phenol/chloroform extraction, or capture of the DNA onto a silica matrix in the presence of high salt concentration.

The target nucleic acid may be single-stranded and may be derived from either the upper or lower strand nucleic acids of double stranded DNA, RNA or other nucleic acid molecules. The upper strand of target nucleic acids includes the plus strand or sense strand of nucleic acids. The lower strand of target nucleic acids is intended to mean the minus or antisense strand that is complementary to the upper strand of target nucleic acids. Thus, reference may be made to either strand and still comprise the polymorphic site and a primer may be designed to hybridize to either or both strands. Target nucleic acids are not meant to be limited to sequences within coding regions, but may also include any region of a genome or portion of a genome containing at least one polymorphism. The term genome is meant to include complex genomes, such as those found in animals, not excluding humans, and plants, as well as much simpler and smaller sources of nucleic acids, such as nucleic acids of viruses, viroids, and any other biological material comprising nucleic acids.

The target nucleic acid sequences or fragments thereof contain the polymorphic site(s), or includes such site(s) and sequences located either distal or proximal to the sites(s). These polymorphic sites or mutations may be in the form of deletions, insertions, re-arrangement, repetitive sequence, base modifications, or single or multiple base changes at a particular site in a nucleic acid sequence. This altered sequence and the more prevalent, or normal, sequence may co-exist in a population. In some instances, these changes confer neither an advantage nor a disadvantage to the species or individuals within the species, and multiple alleles of the sequence may be in stable or quasi-stable equilibrium. In some instances, however, these sequence changes will confer a survival or evolutionary advantage to the species, and accordingly, the altered allele may eventually over time be incorporated into the genome of many or most members of that species. In other instances, the altered sequence confers a disadvantage to the species, as where the mutation causes or predisposes an individual to a genetic disease or defect. As used herein, the terms “mutation” or “polymorphic site” refers to a variation in the nucleic acid sequence between some members of a species, a population within a species or between species. Such mutations or polymorphisms include, but are not limited to, single nucleotide polymorphisms (SNPs), one or more base deletions, or one or more base insertions.

Polymorphisms may be either heterozygous or homozygous within an individual. Homozygous individuals have identical alleles at one or more corresponding loci on homologous chromosomes. Heterozygous individuals have different alleles at one or more corresponding loci on homologous chromosomes. As used herein, alleles include an alternative form of a gene or nucleic acid sequence, either inside or outside the coding region of a gene, including introns, exons, and untranscribed or untranslated regions. Alleles of a specific gene generally occupy the same location on homologous chromosomes. A polymorphism is thus said to be “allelic,” in that, due to the existence of the polymorphism, some members of a species carry a gene with one sequence (e.g., the original or wild-type “allele”), whereas other members may have an altered sequence (e.g., the variant or, mutant “allele”). In the simplest case, only one mutated variant of the sequence may exist, and the polymorphism is said to be biallelic. For example, if the two alleles at a locus are indistinguishable (for example A/A), then the individual is said to be homozygous at the locus under consideration. If the two alleles at a locus are distinguishable (for example A/G), then the individual is said to be heterozygous at the locus under consideration. The vast majority of known single nucleotide polymorphisms are bi-allelic—where there are two alternative bases at the particular locus under consideration.

Having now generally described the invention, the same may be more readily understood through the following reference to the following examples, which are provided by way of illustration and are not intended to limit the spirit or scope of the present invention.

### EXAMPLES

Amplification

For a selected panel, amplicons comprising single nucleotide polymorphisms of the panel are prepared from compromised samples by the polymerase chain reaction (PCR) using a DNA polymerase, Amplitaq Gold™ polymerase, that is thermostable, a DNA template, nucleotides, and two specific primers per amplicon so that both DNA strands of fragments in the compromised sample are copied. A multiplex of these primer pairs is generated to allow the amplification of twelve amplicons in one reaction by combining equimolar amounts (10 μM) of each of the twenty four primers. The DNA is amplified by using a three step procedure: Step one: DNA denaturation (94° C.-100° C.) to generate a single stranded template; Step two: annealing of the primers (45° C.-65° C.) using hybridization conditions that guarantee that the primers will bind perfectly matched target sequences; and Step three: extension or DNA synthesis (72° C.). Usually 30-40 cycles of amplification are carried out to yield millions of copies of the amplicons of interest.

Materials needed include 10% bleach, 2 mL microtubes, single channel pipettes (20 μL-1000 μL), twelve channel pipette (2 μL-20 μL), aerosol resistant pipet tips, 384 well PCR plates and film, 10×PCR Buffer II (Orchid Biosciences, Inc.), 25 mM MgCl₂, 2.5 mM dNTP mix, twelve pair primer pool, Amplitaq Gold™ polymerase, sterile distilled or deionized water, sample DNA, thermal cycler, microcentrifuge, and a vortex.

All PCR reagents should be made in a designated pre-PCR laboratory.

Dedicated lab coats and gloves should be worn and work areas should be cleaned with 10% bleach prior to and after any PCR work is done. PCR reaction mixes should be prepared under a hood. Set aside the following stock reagent to thaw: 2.5 mM dNTPs, 10×PCR Buffer 11, primer pool, 25 mM MgCl₂, sterile water, and DNA samples to be amplified. Calculate the amount needed of each reagent for the specified number of samples and record in the appropriate place on the PCR worksheet (calculate enough for 20% extra samples). Different lot number of the same reagent should never be mixed. Prepare the PCR master mix in a 2 mL microtube and record each reagent's lot number on a PCR sheet.

Typical Amplification Reaction Mix

| Reagent | (per plate/460 samples) | | (per sample) | |
|---|---|---|---|---|
| 10X PCR Buffer II | 230 | μL | 0.5 | μL |
| 25 mM MgCl₂ | 460 | μL | 1 | μL |
| 2.5 mM dNTPs | 69 | μL | 0.15 | μL |
| PCR Primer Pool | 11.5 | μL | 0.025 | μL |
| Water | 563.5 | μL | 1.225 | μL |
| Amplitaq Gold ™ | 46 | μL | 0.1 | μL |
| DNA Template | 2 | μL (2 ng | 2 | μL (2 ng |
| | | total/sample) | | total/sample) |
| Total Volume | 5 | μL per sample | 5 | μL per sample |

PCR Plate Setup

Make sure to mark the orientation of the plate and label the plate with the appropriate marker panel and process group. Add three microliters of the PCR mix to each of the wells using the twelve channel pipet. Spin down the plate containing all of the DNA samples and add two microliters of the DNA template using the twelve channel pipet as before. The samples in the DNA plate are loaded in the same location on the PCR plate. Place a sheet of sealing film on the plate and seal it with the roller. Spin down the plate to remove any bubbles and place in a thermocycler.

Typical PCR Amplification Profile

All amplification reaction are performed on an MJ Research Tetrad™ machine. Programs will vary according to the characteristics of the amplification primers. Selection of melting and annealing temperatures for amplification primers of a panel multiplex reaction are simplified by the use of Autoprimer™ software, as described herein, so that one of ordinary skill in the art can select appropriate extension and melting temperatures for thermal cycling without undue experimentation. A preferred thermal cycler is the MJ Research Tetrad® thermal cycler.

Sample Program:

Mode: Calculated
Step 1: 95° C. 5 minutes
Step 2: 95° C. 30 seconds
Step 3: 50° C. 55 seconds
Step 4: 72° C. 30 seconds
Step 5: Goto step 2 for 2 times
Step 6: 95° C. 30 seconds
Step 7: 50° C. 55 seconds +0.2° per cycle
Step 8: 72° C. for 33 seconds
Step 9: Goto step 6 for 18 times
Step 10: 95° C. 30 seconds
Step 11: 55° C. 55 seconds
Step 12: 72° C. 30 seconds
Step 13: Goto step 10 for 8 times
Step 14: 72° C. 7 minutes
Step 15: 4° C. forever Step 16: End

After the multiplexed PCR amplification of 12 amplicons, unincorporated nucleotides and excess primers are removed enzymatically by methods known in the art, such as treatment with Exonuclease 1 and shrimp alkaline phosphatase. Post-PCR treatment is preferably done with a SNP-IT™ Clean-up kit (Orchid Biosciences, Inc.).

SNP-IT Primer Extension Reaction

Extension mix and a pool of 12 allele-specific tagged SNP-IT™ primers are added to the treated reaction mixture. The allele-specific SNP-IT™ primers hybridize to specific amplicons in the multiplex reaction, immediately adjacent to the polymorphic sites. The tagged primers are extended in a two-dye system by incorporation of a fluorescence labeled chain terminator. Two-color detection allows discrimination of the genotype by comparing signals from the two fluorescence dyes. The extended SNP-IT™ primers are then specifically hybridized to one of 12 unique probes arrayed in each well of a 384 SNP-IT™ plate (Orchid Biosciences, Inc.) through tag-probe capture. The SNP-IT™ primer is a single strand DNA containing a template specific sequence attached with a 5′ non-template specific sequence, wherein “tag” refers to the non-template specific sequence that can be captured by a specific probe bound to a glass surface. A specific probe that hybridizes to one tag is bound to the glass surface of every well in a 384 SNP-IT™ plate. The probes bound covalently to the glass surface enable the interrogation of up to 12-plexed nucleic acid reaction products. The SNP-IT™ reaction product into which the tag has been incorporated will hybridize to the corresponding probe bound covalently to the glass surface. After the extension reaction, the extended SNP-IT™ primers are specifically hybridized to one of 12 unique probes arrayed in each well. The arrayed probes capture the extended products and allow for the detection of each SNP allele signal. Stringent washes will remove free dye-terminators and DNA not hybridized to specific probes.

Probes on the glass surface are arranged in 4×4 arrays in each well in a 384-well format. Three positive controls and one negative control are included in each 4×4 array. The top-left location is heterozygous control which has an equimolar mixture of two probes hybridizing to self-extending oligonucleotides that incorporate two dye labeled terminators. The top-right location has probes that specifically hybridize to self-extending oligonucleotides that incorporate blue dye labeled terminators. The bottom-left location has probes that hybridize to self-extending oligonucleotides that incorporate green-dye labeled terminators. The two self-extending oligonucleotides with equimolar concentration are added into the extension mix and extended with dye-labeled terminator in the cycle extension reaction. The bottom-right location has probes that are not self-extending and lack complementarity to any DNA in the reaction. These probes serve as negative controls in each well.

Primer extension primers are suspended in DNase/RNase-free water and grouped in 12-plexes. Each individual SNP-IT™ primer should be prepared at 120 micromolar. Equal volumes of the 12 SNP-IT™ primers are pooled together. Each SNP-IT™ primer has a final concentration of about 10 micromolar in the pool. At low plexing levels, maintain the concentration of each SNP-IT™ primer at 10 micromolar.

For multiplex SNP-IT™ reactions, pool SNP-IT™ primers to make an equal molar mix. Dilute the SNP-IT™ primer pool 1:100 with molecular biology grade water.

SNP-IT ™ Primers

| | Number of Plates | | | | |
|---|---|---|---|---|---|
| | ⅛ | ¼ | ½ | 1 | 2 |
| SNP-IT ™ Primer | 1.6 μl | 3.2 μl | 6.3 μl | 12.6 μl | 25.2 μl |
| Pool | | | | | |
| H₂0 | 156 μl | 312 μl | 524 μl | 1247 μl | 2495 μl |
| Total Volume | 158 μl | 315 μl | 630 μl | 1260 μl | 2520 μl |

Choose the correct 20× extension mix for the type of SNPs for testing and remove it from −20° C. storage. (For example, T/C SNPs would require a T/C extension mix.)

To prepare extension mixes, calculate the volume of extension mixes needed in the experiments.

Extension Mixes

| | Number of Plates | | | | |
|---|---|---|---|---|---|
| | ⅛ | ¼ | ½ | 1 | 2 |
| 20x Extension Mix | 10.5 μl | 21 μl | 42 μl | 84 μl | 168 μl |
| Extension Mix | 197 μl | 395 μl | 790 μl | 1580 μl | 3160 μl |
| Diluent | | | | | |
| DNA Polymerase | 2.1 μl | 4.2 μl | 8.3 μl | 16.5 μl | 33 μl |
| Total Volume | 210 μl | 420 μl | 840 μl | 1680 μl | 360 μl |

Transfer the diluted SNP-IT™ primer and extension mixes into solution reservoirs for pipetting using multichannel pipette or automatic liquid handling instruments.

Add 3 μl of diluted SNP-IT™ primer pool into corresponding wells of the PCR plates. Spin down the plates with plate centrifuge. Add 4 μl of extension mix prepared described earlier into corresponding wells and mix well.

If the SNP panels are limited (less than or equal to 8), three volumes of diluted SNP-IT™ primer pool can be mixed with four volumes of extension mix. Seven microliters of the extension mix is added into each corresponding well of the PCR plates and mixed by pipetting up and down three times with multichannel pipettor for manual process or by shaking for automatic liquid handling.

Spin down and seal the PCR plates. Thermalcycle using the following program in an MJ Thermalcycler (or equivalent).
Step 1. 96° C. for 3:00 minutes
Step 2. 94° C. for 0:20
Step 3. 40° C. for 0:11
Step 4. Loop steps 2 and 3, 25 times
Step 5. 4° C. final hold temperature
   Note: This program has been optimized for use in a MJ Research Tetrad™. The program may need to be modified for use with a thermalcycler with different heating and cooling rates. The assay may be interrupted at this point. Seal and store SNP-IT™ plate at −20° C. Ensure that plate is thoroughly sealed to avoid evaporation of samples.
   Preparation of SNP-IT Plate

Dilute UHT Prewash solution (20× stock supplied) to 1× with DI H₂O. Wash the SNP-IT™ plate supplied in UHT Core kit ATM, three times with 1×UHT prewash buffer, supplied in the kit. An additional aspirating step should be included to dry the plates. Note: 50 μl/well should be used for each wash if dispensing and aspirating are applied concurrently. The aspiration tip should be close to the glass surface and the edge of the wall.

Preparation of Hybridization Solution

a. Determine the total number plates to be analyzed (regardless of extension mix type or allele reaction).

b. The UHT core kit contains 95 ml of hybridization buffer and 5.5 ml of hybridization additives, enough for processing 10 PCR plates assuming the user processes an average of 2 plates in each run.

c. 550 μl of hybridization additive is mixed well with 9.45 ml of hybridization solution for 2 PCR plates.

d. Add 8 μl of the hybridization solution described previously into each well of the PCR plates and mix well. Transfer 8 μl of the solution from the PCR plates into corresponding well on glass SNP-IT plates.

It is recommended to wash the tips with 3 N NaCl and water between transfers or use new tips for each transfer, to eliminate cross contamination.

Hybridization

After transferring 2 plates, the glass SNP-IT™ plates are placed into a humidified oven (or a covered tray humidified with wet paper towel in an oven) at 42° C. Incubate the plates for 2 hours (+/−15 minutes). It is recommended to process 2-plate batches for a 2 to 12 plates run and 5-plate batches for a 13 to 30 plate run. The run should be staggered for efficient timing.

SNP-IT™ Reaction Wash

Prepare washing solution by mixing 25 ml wash solution 1.575 L of DI H₂O. 50 ml of wash buffer is supplied in the UHT core kit, enough to process 10 PCR plates. After hybridization is complete, wash the SNP-IT™ plates 3 times with washing solution.

Warm-up the SNPstream™ UHT system and input experiment information in UHTPlateExplore™. Verify that you have entered the pre-run data into the UHTPlateExplorer™.

Completely dry the SNP-IT™ plates using the vacuum with a 1 ml pipet tip connected. Cut the tip so it does not touch the glass surface. The cut end should have an aperture bigger than the well. This step may be eliminated if there is an efficient aspiration step at the end of the washing. It is important to note that wet wells increase the background images. Turn on vacuum source and vacuum the wells by rows or columns. Plates are ready to be imaged on the SNPstream™ UHT System. Store SNP-IT™ plates in a dark box, if there is a delay before imaging.

Panels

Thirteen separate panels of about 12 single nucleotide polymorphisms per panel were selected in accordance with the methods of the invention. Each panel member was a T/C single nucleotide polymorphism. These panels were used to screen a variety of samples of compromised nucleic acids.

The amplification primers and SNP-IT™ primers are listed for panels 5 through 17 below. Compromised nucleic acid samples included samples from a building collapse and fire (sample set A), forensic samples from a medical examiner's office (sample set B) and other compromised samples (sample set C) listed in Table 8.

In an attempt to demonstrate proof of principle for this technology nucleic acid samples recovered from a variety of compromised bones, tissues, and other biological samples were genotyped in accordance with the present invention employing a number of panels. Table 1 shows genotypes of compromised nucleic acids of sample set A, run with Panel 5. Table 2 shows genotypes of compromised nucleic acids of sample set A and sample set B run with Panel 6. Table 3 shows genotypes of compromised nucleic acids of sample set C. Table 4 shows genotypes of compromised nucleic acids of sample set C run with Panel 8. Table 5 shows genotypes of compromised nucleic acids of sample set C with Panel 11. Table 6 shows genotypes of compromised nucleic acids of sample set C run with Panel 9. Table 7 shows genotypes of compromised nucleic acids of sample set C run with Panel 10. These data demonstrate the ability of these SNP markers to provide useable genetic information for the purpose of identification.

Table 8 shows Panels 12-17 tested on compromised nucleic acid samples. The results were compared to STR genotyping methods. The comparison in Table 8 establishes that genotyping using panels in accordance with the present invention produced reliable results.

Table 9 shows Panels 12-17 tested on compromised nucleic acid samples. The results show SNPs successfully identified using panels in accordance with the present invention. Table 9 establishes that genotyping using panels in accordance with the present invention produced reliable results.

Table 10 shows Panels 12-17 tested on compromised nucleic acid samples. The results show SNPs successfully identified using panels in accordance with the present invention. Table 10 establishes that genotyping using panels in accordance with the present invention produced reliable results.

Table 11 summaries results from a 44 person study of 24,640 possible genotypes using Panels 12-17 tested on compromised nucleic acid samples. Shown are amounts of DNA used, number of SNPs tested and failures (FL). The results establish that genotyping using panels in accordance with the present invention produced reliable results.

Validation Assay

A validation assay was carried out for 1,560 samples from a building collapse. The protocols for the validation assay are described below.

This assay has been developed using SNP-IT™ technology by taking advantage of the ability for DNA Polymerase to incorporate dye labeled terminators, thus allowing single-base primer extension. Using this technology one can detect single nucleotide polymorphisms (SNP's) by using different dye teminators to distinguish genotypes. After the multiplexed PCR amplification of twelve amplicons, unincorporated nucleotides and primers are removed enzymatically. Extension mix and pool of 12 allele-specific tagged SNP-IT primers are added to the treated PCR. These SNP-IT™ primers hybridize to specific amplicons in the multiplex reaction, one base 3′ of the SNP sites. The tagged primers are extended in a two-dye system, by incorporation of a fluorescence labeled chain-terminating nucleotide. Two-color detection allows discrimination of the genotype by comparing signals from the two fluorescence dyes. The extended SNP-IT™ primers are then specifically hybridized to one of 12 unique probes arrayed in each well. The arrayed probes capture the extended products and allow for the detection of each SNP allele signal.

Assay Protocol

1. Turn on the UHT™ system and related computers.
2. Prepare and place the Correction Plate as the first plate to run.
3. Obtain a new 384-well PCR plate to transfer 5 μL of PCR product from the initial 20 μL PCR plate (source plate):
   a. Quick spin all source plates to be used prior to transfer process. Thaw first if necessary.
   b. Label the new plate with the same information as the source plate (i.e. batch number, panel number, initials, etc.).
   c. Use multichannel pipetter to transfer 5 μL of PCR product from the source plate to the new plate. After completing transfer for entire plate, seal both plates. Store the remaining 15 μL sample plates at −20° C. for re-testing if necessary.
   d. Quick spin the 5 μL plates and do a visual inspection to make sure all samples were transferred properly. If no problems are observed, proceed to the next step, otherwise document problem and notify supervisor.

4. Prepare the Exo/SAP for the SNP-IT™ clean up reaction using the volume calculations:

| | Number of Plates | | | | |
|---|---|---|---|---|---|
| | 2 | 4 | 6 | 8 | 10 |
| Exo/SAP | 101 μl | 202 μl | 303 μl | 404 μl | 505 μl |
| Exo/SAP | 2419 μl | 4838 μl | 7257 μl | 9676 μl | 12095 μl |
| Buffer | | | | | |
| Total | 2.520 ml | 5.040 ml | 7.560 ml | 10.080 ml | 12.600 ml |
| Volume | | | | | |

5. Mix well and transfer to a clean reagent trough.
6. Add 3.0 μl of Exo/SAP mixture to each well of a 384-well PCR plate.
7. Seal plate and quick spin the plate. Be sure to visually check every well to insure that each well received Exo/SAP in an equal amount.
8. Run the Exo/SAP program that cycles the plate from 37° C. for 30 minutes then 10 minutes at 96° C.
Note: This program is optimized for use in the MJ Research Tetrad.
9. Thaw the SNP-IT™ Primer Pool on ice while the Extension Mix is made.
10. Choose the correct 20× Extension Mix for the type of SNPs that are being tested.

11. Prepare the Extension Mix using the following calculations:

| | Number of Plates | | | | |
|---|---|---|---|---|---|
| | ⅛ | ¼ | ½ | 1 | 2 |
| 20x Extension Mix | 10.5 μl | 21 μl | 42 μl | 84 μl | 168 μl |
| Extension Mix Diluent | 197 μl | 395 μl | 790 μl | 1580 μl | 3160 μl |
| Extension Enzyme | 2.1 μl | 4.2 μl | 8.3 μl | 16.5 μl | 33 μl |
| Total Volume | 209.6 μl | 420.2 μl | 840.3 μl | 1680.5 μl | 3361 μl |

12. Dilute the SNP-IT™ Primer Pool using the following calculations:

| | Number of Plates | | | | |
|---|---|---|---|---|---|
| | ⅛ | ¼ | ½ | 1 | 2 |
| SNP-IT ™ Primer | 1.6 μl | 3.2 μl | 6.3 μl | 12.6 μl | 25.2 μl |
| Pool | | | | | |
| H₂O | 156 μl | 312 μl | 524 μl | 1247 μl | 2495 μl |
| Total Volume | 157.6 μl | 315.2 μl | 530.3 μl | 1259.6 μl | 2520.2 μl |

13. Transfer the diluted SNP-IT™ primers and extension mixes into reagent troughs for pipetting using multichannel pipettes
14. Add 3 μl of diluted SNP-IT™ primer pool into corresponding wells of the PCR plates. Spin down the plate quickly. Be sure to visually check every well to insure that each well received SNP-IT™ primer pools in an equal amount.
15. Add 4 μl of extension mix into the corresponding wells and mix well by pipetting up and down.
16. Seal the plate well and spin them down. Visually check to make sure each well received the appropriate amount of liquid.
17. Place the plates in the thermalcycler and run the following program:
   Step 1—96° C., 3:00
   Step 2—94° C., 00:20
   Step 3—40° C., 00:11
   Step 4—Loop steps 2 and 3, 25 times
   Step 5—4° C. final hold
      Note: This program is optimized for use in the MJ Research Tetrad thermalcyler. The assay may be stopped at this point. Seal and store the SNP-IT™ plate at −20° C. Be sure that the plate is thoroughly sealed to avoid evaporation of samples.
18. Dilute 20×UHT™ prewash solution to 1× with sterile water.
19. Wash the SNP-IT™ plate three times with 1×UHT™ prewash buffer. Dry the plates by aspirating with the plate washer.
20. Prepare the hybridization solution in a 15 ml or 50 ml conical tube by adding 550 μl of hybridization additive to 9.45 ml of hybridization solution. Mix well by inversion.
21. Add 8 μl of the hybridization solution to each well of the PCR plate and mix well by pipetting up and down. Then transfer 8 μl of the solution in each well into the corresponding well on the glass SNP-IT plates.
22. Place the glass SNP-IT™ plates into a humidified oven (or a covered tray humidified with wet paper towel in an oven) at 42° C. Incubate the plates for two hours. If you are running many plates, try to stagger them in batches for efficient timing.
23. Prepare stringent wash solution by mixing 25 ml of wash solution to 1.575 L of water (1:64).
24. After hybridization is complete, wash the SNP-IT™ plates three times with stringent wash solution.
25. At this time warm up the UHT™ system and input pre-run information into the UHTPlateExplorer™ software.
26. Remove the SNP-IT™ plates from the oven and completely dry them using a vacuum manifold with tubing connected and a 1 ml pipet tip inserted into the tubing. Cut the pipet tip so it does not touch the glass surface. The cut end should have an aperture bigger than the well. Note: It is extremely important that the plates are perfectly dry. Any remaining liquid increases background images picked up by the laser and could interfere with genotype calling.
27. The plates are ready for imaging on the UHT™ system. Store the plate in a dark place if there will be any delays before imaging.

Using panels 12-17, 1560 tissue samples recovered from a disaster site were tested according to the assay protocol outlined above. The results establish that greater than 50% of the compromised tissue specimens recovered from a disaster site produced genotypes with more than 40 SNPs. These results would likely yield identification indices exceeding 1 in 10⁹.

Summary of Results from Validation Study
(n = 1560)

| No. of SNPs | No. of | |
|---|---|---|
| Working | samples working | Percentage |
| >60 | 643 | 41.22 |
| >50 | 768 | 49.23 |
| >40 | 859 | 55.06 |
| >30 | 947 | 60.71 |
| >20 | 1038 | 66.54 |
| 0, 1, or 2 Failures | 457 | 29.29 |

Bulk Reagent Protocol

Amplification can be carried out using bulk reagents. A typical reaction mixture for carrying out amplifications in 5 microliter and 20 microliter volumes is provided below:

| Reagent | 5 μl Mix | | 20 μl Mix | |
|---|---|---|---|---|
| 10X PCR Buffer II | 0.5 | μl | 3.0 | μl |
| 25 mM MgCl₂ | 1.0 | μl | 6.0 | μl |
| 2.5 mM dNTPs | 0.15 | μl | 0.9 | μl |
| PCR Primer Pool | 0.025 | μl | 0.15 | μl |
| Water | 1.225 | μl | 7.35 | μl |
| AmpliTaq Gold | 0.1 | μl | 0.6 | μl |
| DNA template | 2.0 | μl | 2.0 | μl |
| Pfu enzyme | 0 | | 0.06 | μl |
| Total volume | 5.0 | μl | 20.0 | μl |

Primer Sequences

The sequences of the amplification and identification primers are provided below.

SEQ. ID NO.

PANEL 5 PCR PRIMER SEQUENCES

| | | |
|---|---|---|
| 61955up | tagtttacctctacttcctttcttatattactc | 1 |
| 61955Lo | cacttattttggaaagtggaatc | 2 |
| 195849up | taaggcagccacgggttg | 3 |
| 195849Lo | catgtatgcctgagtgttactgc | 4 |
| 195869up | cagaacacgtgaagactgaa | 5 |
| 195869Lo | catactgaacacatactaatgcagtaatt | 6 |
| 148193up | tatatttcttttcatgagttttgtgag | 7 |
| 148193Lo | cacctgtaatccccccca | 8 |
| 238355up | acttccctgtctggttactcc | 9 |
| 238355Lo | caatgtacagcttgaggacttg | 10 |
| 63635up | tctctccctccccacctc | 11 |
| 63635Lo | gagaacttggcagctccat | 12 |
| 863949up | tatagatgccatcagctcctc | 13 |
| 863949Lo | gaagtgtttctaagcacctgtg | 14 |
| 211489up | actgcatgtgtcagtttcagtc | 15 |
| 211489Lo | gatgagtgaagccactgaagg | 16 |
| 206538up | attttccggagtcagggtc | 17 |
| 206538Lo | gacagccaggctcaagag | 18 |
| 233357up | atttctaccgttactgtcttcttacc | 19 |
| 233357Lo | gaagtcatgctaggctattttaaaga | 20 |
| 207845up | attccatcctgtgctagatgc | 21 |
| 207845Lo | gcactttaataatttggccaga | 22 |
| 231480up | taatatttagagagcagcaaggaca | 23 |
| 231480Lo | cttcttcacccttttcccc | 24 |

PANEL 5 SNP PRIMER SEQUENCES

| | | |
|---|---|---|
| 84760 | acgcacgtccacggtgatttatcagctcctcagatgxgcxcctgact | 25 |
| 195849 | ggatggcgttccgtcctattcagccacgggttgccttctgtaact | 26 |
| 195869 | cgtgccgctcgtgatagaatggtccagaacacgtgaagactgaat | 27 |
| 148193 | agcgatctgcgagaccgtatgagggtattccccaaaxctctgtgttt | 28 |
| 238355 | gcggtaggttcccgacatattggttactccactataaaaxattcatc | 29 |
| 63635 | ggctatgattcgcaatgctttctccctccccacctcctcttgtcc | 30 |
| 863949 | agggtctctacgctgacgatatcagctcctcagatgxgcxcctgact | 31 |
| 211489 | gtgattctgtacgtgtcgcctttcagtcactcattcctttcttcc | 32 |
| 206538 | gacctgggtgtcgatacctaagggtcgggggttctxcxtgttcatct | 33 |
| 233357 | agatagagtcgatgccagctccttcagaagaactcacaaaatacc | 34 |
| 207845 | agagcgagtgacgcatactatgtgctagatgctgxagttgtccttca | 35 |
| 231480 | cgactgtaggtgcgtaactcatttagagagcagcaaxgacattcctc | 36 |

PANEL 6 PCR PRIMER SEQUENCES

| | | |
|---|---|---|
| 63836-U1up | tgcctttcctccagggtc | 37 |
| 63836-U1low | gaaattactgagctcctctggt | 38 |
| 60676-U2up | tgaattgattcaaggggatatatta | 39 |
| 60676-U2low | catattcctctcttgttctctaaacac | 40 |
| 58091-U3up | ggcagtttctttttctctctctc | 41 |
| 58091-U3low | ctcatttattatggtagacaatccc | 42 |
| 169509-U4up | taggagagaatgccagtgtg | 43 |
| 169509-U4low | gttgattggccaggtgga | 44 |
| 238155-U5up | ttgatggcaagaggtaactca | 45 |
| 238155-U5low | gattcaatccaccaaacttactattt | 46 |
| 201688-U6up | aagtaacctggcctctctgag | 47 |
| 201688-U6low | gtgagccaggcattcttg | 48 |
| 57849-U7up | caactcccagtggagagg | 49 |
| 57849-U7low | gataaggcttctgaggtgtgaa | 50 |
| 56915-U8up | tcctcggttgcttctctatc | 51 |
| 56915-U8low | cttgtcaggagtcaacagctt | 52 |
| 56608-U9up | tggtgtggagccaactgg | 53 |
| 56608-U9low | gtctatgaggttgagtctcccc | 54 |
| 68532-U10up | aacttttctcaactactgtttgtgac | 55 |
| 68532-U10low | catttgggtgtaggcggt | 56 |
| 61500-U11up | tttttgccagttgtgtatttttatc | 57 |
| 61500-U11low | caccagtacatactgggcact | 58 |
| 66026-U12up | atttttagagtgaaaggctgct | 59 |
| 66026-U12low | cataagtaaaagaaataagtctcccaa | 60 |

PANEL 6 SNP PRIMER SEQUENCES

| | | |
|---|---|---|
| 63836 | acgcacgtccacggtgatttcaggctgcctttcctccagggtcca | 61 |
| 60676 | ggatggcgttccgtcctatttatattaaattagaatgttgacctc | 62 |
| 58091 | cgtgccgctcgtgatagaatcxctctctttcttcccatagag | 63 |
| 169509 | agcgatctgcgagaccgtattgccagtgtggctcatcaggacatc | 64 |
| 238155 | gcggtaggttcccgacatatatggcaagaggtaactcaa | 65 |
| 201688 | ggctatgattcgcaatgcttctctctgagattcagtttxcacacctg | 66 |
| 57849 | agggtctctacgctgacgatctggaccaacxcxcagtggagagggta | 67 |
| 56915 | gtgattctgtacgtgtcgcccttctctatcataagcacaatg | 68 |
| 56608 | gacctgggtgtcgatacctacaactgggaggagggaaatgagaac | 69 |
| 68532 | agatagagtcgatgccagctttgtgacaacaatacaccaagtacc | 70 |
| 61500 | agagcgagtgacgcatactagtgtatttttatctcatttatccca | 71 |
| 66026 | cgactgtaggtgcgtaactcccatttttagagtgaaaggctgctc | 72 |

PANEL 7 PCR PRIMER SEQUENCES

| | | |
|---|---|---|
| 221499-UP | tttcacaattattatatcagcgaagaac | 73 |
| 221499-LO | ttgatataattaacaaagtacctgaggat | 74 |
| 89446-UP | tttgataagataaattgaattgcaatc | 75 |
| 89446-LO | ccaggaaattatcattcaggaaga | 76 |
| 229291-LO | ctaactgggcatttcaaaataagct | 77 |
| 229291-UP | catctcgtaaagaaaaaaacacatc | 78 |
| 83031-LO | cagattaygctgaatcatgtacactg | 79 |
| 83031-UP | tctggccagcattccagc | 80 |
| 226119-LO | tctaaattgagtcaagatatagaggctttc | 81 |
| 226119-UP | gaactgacattaataatcaatgtacttaca | 82 |
| 60409-UP | tgcaggtgcaatgtttattagctc | 83 |
| 60409-LO | gtatgggaaacttaatcttgtatagtaactt | 84 |
| 220990-UP | acagtaatgagtatagctgtaaattagttatg | 85 |
| 220990-LO | aatatgttttagattcagatttataatttcc | 86 |
| 63527-UP | taccactgtttcctcctttctttct | 87 |
| 63527-LO | atttgccctaggattgagctaac | 88 |
| 230299-LO | tgcaatttgttttcacgtattcg | 89 |
| 230299-UP | cacaggcctggaaagggata | 90 |
| 58040-LO | ygaaaggaaaacctagagagagatt | 91 |
| 58040-UP | gaaacagaaagcgccaaaga | 92 |
| 231480-UP | ctaatatttagagagcagcaaggac | 93 |
| 231480-LO | cttcttcacccttttcccca | 94 |
| 62059-UP | tgataagctacaagttcaaatatactaaac | 95 |
| 62059-LO | gacatagagccagattctaccagg | 96 |
| | | 97 |

PANEL 7 SNP PRIMER SEQUENCES

| | | |
|---|---|---|
| 221449 | acgcacgtccacggtgattttatcagcgxagaacacttcagttgtaa | 98 |
| 89446 | ggatggcgttccgtcctatttgcaatcattttctgaagtttctta | 99 |
| 229291 | cgtgccgctcgtgatagaataaaacxcatcatagcaatctgtgaata | 100 |
| 83031 | agcgatctgcgagaccgtatattccagcxaagctttacttttgataa | 101 |
| 226119 | gcggtaggttcccgacatattaataatcaatxtacxtacataatata | 102 |
| 60409 | ggctatgattcgcaatgctttgtttattagctcgtttatcttcca | 103 |
| 220990 | agggtctctacgctgacgatatagctgtaaattagtxatgatataac | 104 |
| 63527 | gtgattctgtacgtgtcgccactgtttcctcctttctttctctct | 105 |
| 230299 | gacctgggtgtcgatacctaaggcctggaaagggaxattgtgagata | 106 |
| 58040 | agatagagtcgatgccagctagcgccaaagaacagagtagaacaa | 106 |
| 62059 | agagcgagtgacgcatactatacaaxttcaaatatactaaactattc | 108 |
| 231480 | cgactgtaggtgcgtaactcatttagagagcagcaaxgacattcctc | 109 |

PANEL 8 PCR PRIMER SEQUENCES

| | | |
|---|---|---|
| 56763-UP | cgaattttgtgtaggcagcct | 110 |
| 56763-LO | tctacagaggtagatagaattgaatagaag | 111 |
| 61955-UP | tacctctacttcctttcttatattactctt | 112 |
| 61955-LO | gtggatgcaggtcacttattttg | 113 |
| 204593-UP | cacagaatgtgcacagagattgac | 114 |
| 204593-LO | gacattgtacatgatgctgcttag | 115 |
| 65068-UP | ctggaattcttccttctaggtgta | 116 |
| 65068-LO | cttccctaaggctacacttatatattaa | 117 |
| 114977-UP | tgctactaagtctcagatcaattctg | 118 |
| 114977-LO | caataatatgtgtttgttagatcaatacag | 119 |
| 148193-LO | tggctcacacctgtaatccc | 120 |
| 148193-UP | catgagttttgtgagggtattcc | 121 |
| 66158-UP | cttacagataagagaatagaataacaaattac | 122 |
| 66158-LO | gaactgttgtgatattgtggaaaga | 123 |
| 69003-UP | aaaatacctttaacacctatttagtgtc | 124 |
| 69003-LO | ggaaacattttgtaaaaaatcaagta | 125 |
| 63811-UP | tcctaaaccaatcccaggg | 126 |
| 63811-LO | gctcctcctattacctgcaaat | 127 |
| 860850-UP | catgcatccgtccatggg | 128 |
| 860850-LO | atttcctgaatgactgtgtcca | 129 |
| 63189-UP | atccgtccatgggccact | 130 |
| 63189-LO | gctatttcctgaatgactgtgtcc | 131 |
| 126922-UP | gtgctttgataagactgtgatcatcac | 132 |
| 126922-LO | gctgcatgggtccatttgt | 133 |

PANEL 8 SNP PRIMER SEQUENCES

| | | |
|---|---|---|
| 61955 | acgcacgtccacggtgatttcttcctttcttatattactcttttc | 134 |
| 65068 | ggatggcgttccgtcctattttcttccttctaggtgtxtatctatac | 135 |
| 114977 | cgtgccgctcgtgatagaattaagtxtxaxatcaatxctgagaaaga | 136 |
| 148193 | agcgatctgcgagaccgtatgagggtattccccaaaxctctgtgttt | 137 |
| 66158 | gcggtaggttcccgacatatgagaatagaataacaaxttacttga | 138 |
| 56763 | ggctatgattcgcaatgcttttgtgtaggcagccttttagctctt | 139 |
| 69003 | agggtctctacgctgacgatatacctttaaxacctatttagtgtctt | 140 |
| 63811 | gtgattctgtacgtgtcgccaatcccaggggattxcagggttgca | 141 |
| 860850 | gacctgggtgtcgatacctatccgtccatggxccacxcgccgagaca | 142 |
| 63189 | agatagagtcgatgccagcttccgtccatggxccacxcgccgagaca | 143 |
| 126922 | agagcgagtgacgcatactatgtgatcatcacagcaggacagtat | 144 |
| 204593 | cgactgtaggtgcgtaactcgaatgtgcacagagattgactccac | 145 |

PANEL 9 PCR PRIMER SEQUENCES

| | | |
|---|---|---|
| 56593-UP | cagagtggagagtcacaaaatgg | 146 |
| 56593-LO | aatcccttgacactggataacca | 147 |
| 217856-UP | cctctttctctctcctgatctgtctat | 148 |
| 217856-LO | gatggggtgtgaatatgtatacaga | 149 |
| 231735-UP | ctctattatttataaagggcagaatgag | 150 |
| 231735-LO | gcctgtctgtatctctctccttc | 151 |
| 81917-UP | gctctttcatctgatgccatga | 152 |
| 81917-LO | gatataggagtaatctgacagcagg | 153 |
| 62684-UP | taacacaaagaaagtatgcttttgca | 154 |
| 62684-UP | gtatgtggatgaaaatctcgcac | 155 |
| 241554-UP | gtgataataaaatttttgtgcctga | 156 |
| 241554-LO | catttgtttcacctgtgttcttaata | 157 |
| 126264-UP | ggataatgttctccgtaaggtttatac | 158 |
| 126264-LO | gagaaacaagcttgcccttaacta | 159 |
| 224922-UP | caaggaaaacttacataatcacagc | 160 |
| 224922-LO | gaaatataaaagctccacaaatagga | 161 |
| 81081-UP | aaagtaggcaatactgaagagtcatac | 162 |
| 81081-LO | gttcaattggcttggaagttatacc | 163 |
| 66561-LO | acttggatttaccctcattgatg | 164 |
| 66561-UP | cttcctctttggtttctgcttttaat | 165 |
| 63799-UP | gtgcccagctccctaatttct | 166 |
| 63799-LO | ctcttgtgactttcattaactatcttca | 167 |
| 119770-UP | agcctggctggaaatgaag | 168 |
| 119770-LO | cttctaccctcctgtacctgattta | 169 |

PANEL 9 SNP PRIMER SEQUENCES

| | | |
|---|---|---|
| 56593 | acgcacgtccacggtgattttggagagtcacaaaatgxcccttatta | 170 |
| 217856 | ggatggcgttccgtcctatttttctctctcctxatctgtctatcaaa | 171 |
| 231735 | cgtgccgctcgtgatagaattttataaagggcagaatgaggatta | 172 |
| 81917 | agcgatctgcgagaccgtattcatctgatgccatgagaaagc | 173 |
| 62684 | gcggtaggttcccgacatatagaaagtatxcxttxgcaaaaggtcca | 174 |
| 241554 | ggctatgattcgcaatgctttaataaaatttttgtgcxtgaggtata | 175 |
| 126264 | agggtctctacgctgacgatttctccgtaaggtttxtacattgacta | 176 |
| 224922 | gtgattctgtacgtgtcgcccataatcacagcttttttctcccaa | 177 |
| 81081 | gacctgggtgtcgatacctataggcaatactgaagagtcatacaa | 178 |
| 66561 | agatagagtcgatgccagctgxttctgctxttaatacaaaaccag | 179 |
| 63799 | agagcgagtgacgcatactaagctcxctaatttcttgatggg | 180 |
| 119770 | cgactgtaggtgcgtaactctggctggaaatgaaggaaaggaaag | 181 |

PANEL 10 PCR PRIMER SEQUENCES

| | | |
|---|---|---|
| 63836-LO | ctctggtgcccgacagc | 182 |
| 63836-up | gcatcaggctgcctttcct | 183 |
| 58091-UP | ctttttctctctctctttcttccc | 184 |
| 58091-LO | gctcatttattatggtagacaatcc | 185 |
| 68909-UP | gagtgttgggaagagagaccttc | 186 |
| 68909-LO | gctatgtggacagacccatctg | 187 |
| 238155-UP | ggtacttgatggcaagaggtaact | 188 |
| 238155-LO | aaacttactatttggatagagtgcttt | 189 |
| 201688-LO | ctgtgagccaggcattcttg | 190 |
| 201688-UP | caagtaacctggcctctctgagat | 191 |
| 57849-UP | gctggaccaactcccagtg | 192 |
| 57849-LO | gtgaatatctctcctttctctggg | 193 |
| 56915-UP | cctcggttgcttctctatcataa | 194 |
| 56915-LO | cttgtcaggagtcaacagcttc | 195 |
| 56608-LO | aggttgagtctcccccgtg | 196 |
| 56608-UP | gtggagccaactgggagga | 197 |
| 68532-UP | cttttctcaactactgtttgtgaca | 198 |
| 68532-LO | ccatttgggtgtaggcgg | 199 |
| 61500-UP | ttgccagttgtgtatttttatctca | 200 |
| 61500-LO | taacttaagcccaccagtacatact | 201 |
| 66026-UP | cccatttttagagtgaaaggctg | 202 |
| 66026-LO | taagtctcccaaggtggatacatg | 203 |
| 60676-UP | gattcaaggggatatattaaattagaat | 204 |
| 60676-LO | caagttcatattcctctcttgttctc | 205 |

PANEL 10 SNP PRIMER SEQUENCES

| | | |
|---|---|---|
| 63836 | acgcacgtccacggtgatttcaggctgcctttcctccagggtcca | 206 |
| 60676 | ggatggcgttccgtcctatttatattaaattagaatgttgacctc | 207 |
| 58091 | cgtgccgctcgtgatagaatcxctctctttcttcccatagag | 208 |
| 68909 | agcgatctgcgagaccgtattgttxggxagagagaccttccattcat | 209 |
| 238155 | gcggtaggttcccgacatatatggcaagaggtaactcaatca | 210 |
| 201688 | ggctatgattcgcaatgcttctctctgagattcagtttxcacacctg | 211 |
| 57849 | agggtctctacgctgacgatctggaccaacxcxcagtggagagggta | 212 |
| 56915 | gtgattctgtacgtgtcgcccttctctatcataagcacaatg | 213 |
| 56608 | gacctgggtgtcgatacctacaactgggaggagggaaatgagaac | 214 |
| 68532 | agatagagtcgatgccagctttgtgacaacaatacaccaagtacc | 215 |
| 61500 | agagcgagtgacgcatactagtgtatttttatctcatttatccca | 216 |
| 66026 | cgactgtaggtgcgtaactcccatttttagagtgaaaggctgctc | 217 |

PANEL 11 PCR PRIMER SEQUENCES

| | | |
|---|---|---|
| 212605-UP | gcctgcttcccctttatcct | 218 |
| 212605-LO | tcttatctcccatcttcctctacac | 219 |
| 220875-UP | ctggcaatctgggcacc | 220 |
| 220875-LO | cccaagtccacacacaaattat | 221 |
| 65882-UP | gtatactaaagagtctaagtttttgcctaa | 222 |
| 65882-LO | cttccctttttccttccctt | 223 |
| 57575-UP | tgaatagtctttggtctgagcct | 224 |
| 57575-LO | aggcagagtcttatctgggaca | 225 |
| 66683-UP | cagagaattggagttggctgg | 226 |
| 66683-LO | aggaggtagcagtcacactgattc | 227 |
| 214674-UP | gacttccgattgtgaggctg | 228 |
| 214674-LO | cctccttttattcttgctcatagc | 229 |
| 248007-UP | agctcactggatgcaagagtagt | 230 |
| 248007-LO | caagtggataagatgacccattc | 231 |
| 63804-UP | gatatacaggggaaacgggct | 232 |
| 63804-LO | cctcaggggggcactttac | 233 |
| 56144-UP | tcaatcttttgatgatgtcctaaga | 234 |
| 56144-LO | ttcagcacagtattctagtattttgtg | 235 |
| 233357-UP | cgttactgtcttcttacccttcag | 236 |
| 233357-LO | ggaagtcatgctaggctattttaa | 237 |
| 206538-UP | agggtcgggggttctgc | 238 |
| 206538-LO | ctacagcctagggacagccag | 239 |
| 60188-UP | aggatgcatgcatgctgg | 240 |
| 60188-LO | ctcagagtatgtgccattgattg | 241 |

PANEL 11 SNP PRIMER SEQUENCES

| | | |
|---|---|---|
| 212605 | acgcacgtccacggtgatttttcccctttatcctcttcgcagcct | 242 |
| 220875 | ggatggcgttccgtcctattatctgggcxccaggcaggtggtcaggc | 243 |
| 65882 | cgtgccgctcgtgatagaatagtctaagtxtttgcctaaaagcagga | 244 |
| 57575 | agcgatctgcgagaccgtattgaatagtctttxgtctgagcctggaa | 245 |
| 66683 | gcggtaggttcccgacatatagagaattggagttggctggagata | 246 |
| 214674 | ggctatgattcgcaatgcttccgattgtgaggctgctgagaaggg | 247 |
| 248007 | agggtctctacgctgacgataagagtagttggggaaaggggctgt | 248 |
| 63804 | gtgattctgtacgtgtcgccatacaggggaaacxggxtccgagcaga | 249 |
| 56144 | gacctgggtgtcgatacctatgatgatgtcctaxgaaataatgactt | 250 |
| 233357 | agatagagtcgatgccagctccttcagaagaactcacaaaatacc | 251 |
| 60188 | agagcgagtgacgcatactagatgcatgcatgctgxcxttgaggaac | 252 |
| 206538 | cgactgtaggtgcgtaactcagggtcgggggttctxcxtgttcatct | 253 |

PANEL 12 PCR PRIMER SEQUENCES

| | | |
|---|---|---|
| 56593-UP | cagagtggagagtcacaaaatgg | 254 |
| 56593-LO | aatcccttgacactggataacca | 255 |
| 217856-UP | cctctttctctctcctgatctgtctat | 256 |
| 217856-LO | gatggggtgtgaatatgtatacaga | 257 |
| 231735-UP | ctctattatttataaagggcagaatgag | 258 |
| 231735-LO | gcctgtctgtatctctctccttc | 259 |
| 81917-UP | acttagcttggttctttgttttctaattaac | 260 |
| 81917-LO | atggaaaggcagatataggagtaatct | 261 |
| 62684-UP | taacacaaagaaagtatgcttttgca | 262 |
| 62684-UP | gtatgtggatgaaaatctcgcac | 263 |
| 241554-UP | gtgataataaaatttttgtgcctga | 264 |
| 241554-LO | catttgtttcacctgtgttcttaata | 265 |
| 126264-UP | ggataatgttctccgtaaggtttatac | 266 |
| 126264-LO | gagaaacaagcttgcccttaacta | 267 |
| 230299-LO | tgcaatttgttttcacgtattcg | 268 |
| 230299-UP | cacaggcctggaaagggata | 269 |
| 224922-UP | caaggaaaacttacataatcacagc | 270 |
| 224922-LO | gaaatataaaagctccacaaatagga | 271 |
| 66561-LO | acttggatttaccctcattgatg | 272 |
| 66561-UP | cttcctctttggtttctgcttttaat | 273 |
| 63799-UP | gtgcccagctccctaatttct | 274 |
| 63799-LO | ctcttgtgactttcattaactatcttca | 275 |
| 119770-UP | agcctggctggaaatgaag | 276 |
| 119770-LO | cttctaccctcctgtacctgattta | 277 |

PANEL 12 SNP PRIMER SEQUENCES

| | | |
|---|---|---|
| 56593 | acgcacgtccacggtgattttggagagtcacaaaatgxcccttatta | 278 |
| 217856 | ggatggcgttccgtcctatttttctctctcctxatctgtctatcaaa | 279 |
| 231735 | cgtgccgctcgtgatagaattttataaagggcagaatgaggatta | 280 |
| 81917 | agcgatctgcgagaccgtattcatctgatgccatgagaaagc | 281 |
| 62684 | gcggtaggttcccgacatatagaaagtatxcxttxgcaaaaggtcca | 282 |
| 241554 | ggctatgattcgcaatgctttaataaaatttttgtgcxtgaggtata | 283 |
| 126264 | agggtctctacgctgacgatttctccgtaaggtttxtacattgacta | 284 |
| 224922 | gtgattctgtacgtgtcgcccataatcacagcttttttctcccaa | 285 |
| 230299 | gacctgggtgtcgatacctaaggcctggaaagggaxattgtgagata | 286 |
| 66561 | agatagagtcgatgccagctgxttctgctxttaatacaaaaccag | 287 |
| 63799 | agagcgagtgacgcatactaagctcxctaatttcttgatggg | 288 |
| 119770 | cgactgtaggtgcgtaactctggctggaaatgaaggaaaggaaag | 289 |

PANEL 13 PCR PRIMER SEQUENCES

| | | |
|---|---|---|
| 63836-UP | gcatcaggctgcctttcct | 290 |
| 63836-LO | ctctggtgcccgacagc | 291 |
| 220875-UP | ctggcaatctgggcacc | 292 |
| 220875-LO | cccaagtccacacacaaattat | 293 |
| 58091-UP | aatacttcatctctgggggca | 294 |
| 58091-LO | gctcatttattatggtagacaatcc | 295 |
| 68909-UP | gagtgttgggaagagagaccttc | 296 |
| 68909-LO | gctatgtggacagacccatctg | 297 |
| 238155-UP | ggtacttgatggcaagaggtaact | 298 |
| 238155-LO | aaacttactatttggatagagtgcttt | 299 |
| 201688-UP | caagtaacctggcctctctgagat | 300 |
| 201688-LO | ctgtgagccaggcattcttg | 301 |
| 57849-UP | gctggaccaactcccagtg | 302 |
| 57849-LO | gtgaatatctctcctttctctggg | 303 |
| 56915-UP | cctcggttgcttctctatcataa | 304 |
| 56915-LO | cttgtcaggagtcaacagcttc | 305 |
| 56608-UP | gtggagccaactgggagga | 306 |
| 56608-LO | aggttgagtctcccccgtg | 307 |
| 68532-UP | cttttctcaactactgtttgtgaca | 308 |
| 68532-LO | ccatttgggtgtaggcgg | 309 |
| 62059-UP | tgataagctacaagttcaaatatactaaac | 310 |
| 62059-LO | gacatagagccagattctaccagg | 311 |
| 66026-UP | cccatttttagagtgaaaggctg | 312 |
| 66026-LO | taagtctcccaaggtggatacatg | 313 |

PANEL 13 SNP PRIMER SEQUENCES

| | | |
|---|---|---|
| 63836 | acgcacgtccacggtgatttcaggctgcctttcctccagggtcca | 314 |
| 220875 | ggatggcgttccgtcctattatctgggcxccaggcaggtggtcaggc | 315 |
| 58091 | cgtgccgctcgtgatagaatcxctctctttcttcccatagag | 316 |
| 68909 | agcgatctgcgagaccgtattgttxggxagagagaccttccattcat | 317 |
| 238155 | gcggtaggttcccgacatatatggcaagaggtaactcaatca | 318 |
| 201688 | ggctatgattcgcaatgcttctctctgagattcagtttxcacacctg | 319 |
| 57849 | agggtctctacgctgacgatctggaccaacxcxcagtggagagggta | 320 |
| 56915 | gtgattctgtacgtgtcgcccttctctatcataagcacaatg | 321 |
| 56608 | gacctgggtgtcgatacctacaactgggaggagggaaatgagaac | 322 |
| 68532 | agatagagtcgatgccagctttgtgacaacaatacaccaagtacc | 323 |
| 62059 | agagcgagtgacgcatactatacaaxttcaaatatactaaactattc | 324 |
| 66026 | cgactgtaggtgcgtaactcccatttttagagtgaaaggctgctc | 325 |

PANEL 14 PCR PRIMER SEQUENCES

| | | |
|---|---|---|
| | | 326 |
| 76268-UP | ctgtttcatttcagcccttttag | 327 |
| 76268-LO | gttatccttagtgagttttctgtctaca | 328 |
| 70371-UP | gcgtcatatggagcctcct | 329 |
| 70371-LO | ctcatctggccttctgtgtcc | 330 |
| 58388-UP | ctgcagttcaggtggctgtt | 331 |
| 58388-LO | cctcgtctccaagggtgtct | 332 |
| 105677-UP | agccattagacctgccaatc | 333 |
| 105677-LO | aatgcagaggccaccagc | 334 |
| 226119-UP | gaactgacattaataatcaatgtacttaca | 335 |
| 226119-LO | tctaaattgagtcaagatatagaggctttc | 336 |
| 63184-UP | ctcaagcactctctcttttcatca | 337 |
| 63184-LO | ggagtccaggtagataggaacactag | 338 |
| 63979-UP | gtgatacacgaaggcagatgat | 339 |
| 63979-LO | gactgtgaatgtacttagccccc | 340 |
| 130240-UP | caacaggaagcgaggcc | 341 |
| 130240-LO | acaaggcaggaccaaggc | 342 |
| 182622-UP | gggcttgtgtgtccacaga | 343 |
| 182622-LO | tgtgtcaggaagaagaagatcaac | 344 |
| 66567-UP | ctgaacccaagaacttcctgat | 345 |
| 66567-LO | tgatgagtatataaccagaaggaacac | 346 |
| 89614-UP | agcagaggatggcagtcacc | 347 |
| 89614-LO | cacctctgttcctgttttctgtta | 348 |
| 219561-UP | cagtactatctcttctttaaagatctgaaa | 349 |
| 219561-LO | acccagctcaagatgctctg | 350 |

PANEL 14 SNP PRIMER SEQUENCES

| | | |
|---|---|---|
| 76268 | acgcacgtccacggtgatttttaggtatagttgattgttttaaga | 351 |
| 70371RT | ggatggcgttccgtcctattgcgtcatatgxagcctxctgggacaag | 352 |
| 58388 | cgtgccgctcgtgatagaatttcaggtggctgtttcagagctcag | 353 |
| 105677 | agcgatctgcgagaccgtatcxattagacctgccaatcxcctggaga | 354 |
| 226119 | gcggtaggttcccgacatattaataatcaatxtacxtacataatata | 355 |
| 63184 | ggctatgattcgcaatgcttcactctctcttttcatcactcatct | 356 |
| 63979 | agggtctctacgctgacgatcacgaaxgcagatxatxacggtcgcct | 357 |
| 130240 | gtgattctgtacgtgtcgccgaagcgaggccxcaggtcaaggtggga | 358 |
| 182622 | gacctgggtgtcgatacctatgtgtcxacagacagtggcgggcttca | 359 |
| 66567 | agatagagtcgatgccagctcaagaactxcctgatatgggaatcaaa | 360 |
| 89614 | agagcgagtgacgcatactacagtcaccctcagagcccagaa | 361 |
| 219561 | cgactgtaggtgcgtaactctgaaagtagaaccaatcaaggctcc | 362 |

PANEL 15 PCR PRIMER SEQUENCES

| | | |
|---|---|---|
| 216327-UP | cagtgggctctatttttttctaactt | 363 |
| 216327-LO | tggtctctcagctatggcctt | 364 |
| 248075-UP | gatcaaaaaagcatgagttcttatta | 365 |
| 248075-LO | cctcactaatggtgacacaacaag | 366 |
| 85187-UP | cccaggcaattaatgagtctg | 367 |
| 85187-LO | gtttatatattaggaacttttaggggag | 368 |
| 225225-UP | ctagacctaaatagtggccctaaat | 369 |
| 225225-LO | ctctactgaagacaaacttagaggaatg | 370 |
| 82031-UP | ttgacatcttcttagattctaaaatcac | 371 |
| 82031-LO | ctgttggcttttaaggtctcc | 372 |
| 60409-UP | tgcaggtgcaatgtttattagctc | 373 |
| 60409-LO | gtatgggaaacttaatcttgtatagtaactt | 374 |
| 221499-UP | tttcacaattattatatcagcgaagaac | 375 |
| 221499-LO | ttgatataattaacaaagtacctgaggat | 376 |
| 168115-UP | tcctgtagcattggaaaactgt | 377 |
| 168115-LO | agaaactggagttactcttgtcaga | 378 |
| 177589-UP | ctgaggaagagtgcagcatactc | 379 |
| 177589-LO | caggcatagggttgggatg | 380 |
| 173632-UP | gactcttcatggccaacacc | 381 |
| 173632-LO | attttgccactagtttttacatctcta | 382 |
| 60188-UP | aggatgcatgcatgctgg | 383 |
| 60188-LO | ctcagagtatgtgccattgattg | 384 |
| 231480-UP | ctaatatttagagagcagcaaggac | 385 |
| 231480-LO | cttcttcacccttttcccca | 386 |

PANEL 15 SNP PRIMER SEQUENCES

| | | |
|---|---|---|
| 216327 | acgcacgtccacggtgatttctatttttttctaacttcagaattt | 387 |
| 248075RT | ggatggcgttccgtcctattgcatgagttcttattattcaccaca | 388 |
| 85187 | cgtgccgctcgtgatagaatgcaattaatgagtctgxtaaaccta | 389 |
| 225225 | agcgatctgcgagaccgtatccctaaatttgtgttaxgcxttcccta | 390 |
| 82031 | gcggtaggttcccgacatattagattctxaaatcactttattcatac | 391 |
| 60409 | ggctatgattcgcaatgctttgtttattagctcgtttatcttcca | 392 |
| 221499RT | agggtctctacgctgacgattatcagcgxagaacacttcagttgtaa | 393 |
| 168115 | gtgattctgtacgtgtcgccaaactgttgttcattttctcaccac | 394 |
| 177589 | gacctgggtgtcgatacctagtgcagcatactcattcacaga | 395 |
| 173632 | agatagagtcgatgccagcttcatggccaacaxcaggtagtcagtat | 396 |
| 60188 | agagcgagtgacgcatactagatgcatgcatgctgxcxttgaggaac | 397 |
| 231480 | cgactgtaggtgcgtaactcatttagagagcagcaaxgacattcctc | 398 |

PANEL 16 PCR PRIMER SEQUENCES

| | | |
|---|---|---|
| 61955-UP | tacctctacttcctttcttatattactctt | 399 |
| 61955-LO | gtggatgcaggtcacttattttg | 400 |
| 65068-UP | ctggaattcttccttctaggtgta | 401 |
| 65068-LO | cttccctaaggctacacttatatattaa | 402 |
| 65882-UP | gtatactaaagagtctaagtttttgcctaa | 403 |
| 65882-LO | cttccctttttccttccctt | 404 |
| 148193-UP | catgagttttgtgagggtattcc | 405 |
| 148193-LO | tggctcacacctgtaatccc | 406 |
| 66158-UP | cttacagataagagaatagaataacaaattac | 407 |
| 66158-LO | gaactgttgtgatattgtggaaaga | 408 |
| 56763-UP | cgaattttgtgtaggcagcct | 409 |
| 56763-LO | tctacagaggtagatagaattgaatagaag | 410 |
| 69003-UP | aaaatacctttaacacctatttagtgtc | 411 |
| 69003-LO | ggaaacattttgtaaaaaatcaagta | 412 |
| 212605-UP | gcctgcttcccctttatcct | 413 |
| 212605-LO | tcttatctcccatcttcctctacac | 414 |
| 860850-UP | catgcatccgtccatggg | 415 |
| 860850-LO | atttcctgaatgactgtgtcca | 416 |
| 235106-UP | gcttttgaaaaaaaataaaattgc | 417 |
| 235106-LO | ggacccatttatagttttttaactttg | 418 |
| 126922-UP | gtgctttgataagactgtgatcatcac | 419 |
| 126922-LO | gctgcatgggtccatttgt | 420 |
| 206538-UP | agggtcgggggttctgc | 421 |
| 206538-LO | ctacagcctagggacagccag | 422 |

PANEL 16 SNP PRIMER SEQUENCES

| | | |
|---|---|---|
| 61955 | acgcacgtccacggtgatttcttcctttcttatattactcttttc | 423 |
| 65068 | ggatggcgttccgtcctattttcttccttctaggtgtxtatctatac | 424 |
| 65882 | cgtgccgctcgtgatagaatagtctaagtxtttgcctaaaagcagga | 425 |
| 148193 | agcgatctgcgagaccgtatgagggtattccccaaaxctctgtgttt | 426 |
| 66158 | gcggtaggttcccgacatatgagaatagaataacaaxttacttga | 427 |
| 56763 | ggctatgattcgcaatgcttttgtgtaggcagccttttagctctt | 428 |
| 69003 | agggtctctacgctgacgatatacctttaaxacctatttagtgtctt | 429 |
| 212605RT | gtgattctgtacgtgtcgccttcccctttatcctcttcgcagcct | 430 |
| 860850 | gacctgggtgtcgatacctatccgtccatggxccacxcgccgagaca | 431 |
| 235106 | agatagagtcgatgccagctaxaaataxaattgcttttgaatactga | 432 |
| 126922 | agagcgagtgacgcatactatgtgatcatcacagcaggacagtat | 433 |
| 206538 | cgactgtaggtgcgtaactcagggtcgggggttctxcxtgttcatct | 434 |

PANEL 17 PCR PRIMER SEQUENCES

| | | |
|---|---|---|
| 228468-UP | cctactttcagatcctgagtcttgt | 435 |
| 228468-LO | gcctctggtgttatttagactcc | 436 |
| 214674-UP | gacttccgattgtgaggctg | 437 |
| 214674-LO | cctccttttattcttgctcatagc | 438 |
| 126243-UP | ccagtgtttgaatgccgct | 439 |
| 126243-LO | gaagcggaggtttcagcag | 440 |
| 207160-UP | tgaatgaattaacaaagtcatggag | 441 |
| 207160-LO | ctctgcccccattccaac | 442 |
| 66683-UP | cagagaattggagttggctgg | 443 |
| 66683-LO | aggaggtagcagtcacactgattc | 444 |
| 211324-UP | tgccacacagtttggagtga | 445 |
| 211324-LO | cattcaatgggggagatgg | 446 |
| 214373-UP | ctggcaggcaagagatgtga | 447 |
| 214373-LO | gactggaaaggaacaaagaggtg | 448 |
| 234217-UP | acagtcatttgtacttacggagcg | 449 |
| 234217-LO | gagcctgcctcaacgagaag | 450 |
| 63404-UP | aggggctargtttggagaagag | 451 |
| 63404-LO | aatgcaaagaccacatctatcaat | 452 |
| 72171-UP | cacctgacctccagcaagag | 453 |
| 72171-LO | ggtgtgtccctgtgtgtagtgg | 454 |
| Amel-2-short-UP | ccagataaagtggtttctcaagtg | 455 |
| Amel-2-short-LO | gggaagctggtggtaggaac | 456 |

PANEL 17 SNP PRIMER SEQUENCES

| | | |
|---|---|---|
| 228468 | acgcacgtccacggtgattttcctgagtcttgttttgacccatga | 457 |
| 214674RT | ggatggcgttccgtcctattccgattgtgaggctgctgagaaggg | 458 |
| 126243 | cgtgccgctcgtgatagaataatgccgctgtgagacaaaggg | 459 |
| 207160 | agcgatctgcgagaccgtataacaaagtcatggagaaatcaactc | 460 |
| 66683 | gcggtaggttcccgacatatagagaattggagttggctggagata | 461 |
| 211324 | ggctatgattcgcaatgctttttgccacacagttxggagtgacccaa | 462 |
| 214373RT | agggtctctacgctgacgatggcaagagatgtgacaggcaagagt | 463 |
| 234217 | gacctgggtgtcgatacctaacttacggagcgctctttgtgagaa | 464 |
| 63404 | agatagagtcgatgccagctrgtttggagaxgagcctacrtcttaac | 465 |
| 72171 | cgactgtaggtgcgtaactctccaxcaagaggaatxcaagaatgcta | 466 |
| Amel-2U8 | gtgattctgtacgtgtcgccgataaagtggtttctcaagtggtcc | 467 |

TABLE 1
GENOTYPES OF COMPROMISED NUCLEIC ACID SAMPLES SET A, RUN WITH PANEL 5 (12 SNPS TOTAL). SAMPLES ARE
HOMOZYGOTES (XX OR YY), HETEROZYGOTES (XY), OR SAMPLES DID NOT TYPE (—) FOR EACH RESPECTIVE SNP.

| | | | | | | | | | | | | | # OF |
|---|---|---|---|---|---|---|---|---|---|---|---|---|---|
| | 84760 | 195849 | 195869 | 148193 | 238355 | 63635 | 863949 | 211489 | 206538 | 233357 | 207845 | 231480 | SNPS SUCCESSFUL |
| DQ 31770 | — | XX | XY | YY | XX | XX | YY | XX | XY | YY | YY | XX | 11/12 |
| DQ 31749 | — | YY | — | YY | — | YY | XX | XX | XX | XY | XY | XY | 9/12 |
| DQ 31965 | — | XY | — | YY | XX | XY | XX | XX | YY | YY | XX | YY | 10/12 |
| DQ | — | YY | YY | XY | XX | XY | YY | XX | YY | YY | YY | XY | 11/12 |
| 232121 | | | | | | | | | | | | | |
| DQ 14700 | — | XY | — | XY | XX | XY | XX | XX | — | YY | YY | XY | 9/12 |
| DQ 14704 | — | XY | — | XY | — | XY | — | — | YY | XY | — | XY | 6/12 |
| DQ 14775 | — | YY | YY | XY | — | YY | YY | XX | YY | XY | YY | XY | 10/12 |
| DQ 12793 | — | XY | — | XY | XX | XY | XY | — | XY | XY | XY | XY | 9/12 |
| DQ 12792 | — | — | — | XX | XX | XY | YY | — | YY | YY | XX | YY | 8/12 |
| DQ 14686 | — | XY | — | XX | XX | XY | XX | — | XY | XY | YY | XY | 9/12 |

TABLE 2
GENOTYPES OF COMPROMISED NUCLEIC ACID SAMPLES SET A AND COMPROMISED NUCLEIC ACID SAMPLES SET B RUN
WITH PANEL 6 (12 SNPS TOTAL). SAMPLES ARE HOMOZYGOTES (XX OR YY), HETEROZYGOTES (XY), OR SAMPLES DID
NOT TYPE (—) FOR EACH RESPECTIVE SNP.

| | | | | | | | | | | | | | # OF SNPS |
|---|---|---|---|---|---|---|---|---|---|---|---|---|---|
| | 63836 | 60676 | 58091 | 169509 | 238155 | 201688 | 57849 | 56915 | 56608 | 68532 | 61500 | 66026 | SUCCESSFUL |
| DQ 12792 | XY | YY | XY | XY | YY | XY | XY | XY | XY | XX | XY | XY | 12/12 |
| DQ 12793 | XY | XY | XY | XY | XY | XY | XY | XY | XY | XY | XY | XY | 12/12 |
| DQ 14686 | XY | XY | XY | XY | XY | XY | XX | YY | XX | XY | XY | XY | 12/12 |
| DQ 14775 | XY | YY | XY | YY | YY | XY | XX | XX | XY | XX | XY | XY | 12/12 |
| DQ 14700 | XY | YY | XY | XY | XX | YY | XY | YY | XX | XY | XY | XY | 12/12 |
| DQ 14704 | XY | XX | YY | XY | XY | XY | YY | XX | XY | XY | XY | XY | 12/12 |
| DQ 231770 | YY | YY | XX | XY | YY | YY | XY | XX | XX | XY | XY | XY | 12/12 |
| DQ 231965 | XY | XY | XY | −Y | XY | XY | XY | XY | XX | YY | YY | XY | 12/12 |
| DQ 232121 | — | — | — | — | — | — | — | — | — | — | — | — | No DNA |
| DQ 231749 | YY | XY | XX | XY | XY | XY | XY | XY | XX | XY | XY | YY | 12/12 |
| DFS 2918034 | — | — | — | — | — | — | — | — | — | — | — | — | 0/12 |
| DFS 294240 | — | — | — | — | — | — | — | — | — | — | — | — | 0/12 |
| DFS 294235 | XY | YY | XX | YY | YY | XY | YY | XY | XX | XY | XY | YY | 12/12 |
| DFS 2918027 | — | — | — | — | — | — | — | — | — | — | — | — | 0/12 |
| DFS 3260001 | — | — | — | — | — | — | — | — | — | — | — | — | 0/12 |
| DFS 3258001 | YY | — | — | — | — | — | — | — | — | — | — | XY | 2/12 |
| DFS MITO | YY | XY | YY | XY | YY | XY | XY | XY | XY | XY | XY | YY | 12/12 |
| DFS HAIR | YY | — | — | — | — | — | XX | — | — | — | — | — | 2/12 |

TABLE 3
PANEL 7 RUN WITH COMPROMISED NUCLEIC ACID SAMPLES SET C. THE SOURCES
OF THE COMPROMISED SAMPLES OF SET C ARE DESCRIBED IN TABLE 8.

| | 221499 | 89446 | 229291 | 83031 | 226119 | 60409 | 220990 | 63527 | 230299 | 58040 | 62059 | 231480 |
|---|---|---|---|---|---|---|---|---|---|---|---|---|
| 3260-1 | — | — | — | — | — | — | — | — | — | — | — | — |
| 3135-4 | YY | — | — | XX | YY | — | — | XY | XX | YY | XX | YY |
| 3135-5 | — | — | XX | — | — | — | — | — | — | — | XX | YY |
| 3135-6 | — | — | — | — | — | — | — | — | — | — | — | — |
| 3106-4 | — | YY | — | — | — | — | — | XY | — | — | YY | YY |
| 3106-2 | — | — | — | — | — | — | — | XX | — | — | — | — |
| 3106-7 | — | — | — | YY | YY | — | — | XX | — | — | YY | XX |

TABLE 4
PANEL 8 RUN WITH COMPROMISED NUCLEIC ACID SAMPLES SET C. THE SOURCES
OF THE COMPROMISED SAMPLES OF SET C ARE DESCRIBED IN TABLE 8.

| | 61955 | 65068 | 114977 | 148193 | 66158 | 56763 | 69003 | 63811 | 860850 | 63189 | 126922 | 204593 |
|---|---|---|---|---|---|---|---|---|---|---|---|---|
| 3260-1 | — | — | — | — | — | — | — | — | — | — | — | — |
| 3135-4 | — | XY | — | XX | — | YY | XX | XY | — | — | YY | — |
| 3135-5 | — | — | — | — | — | — | — | — | — | — | — | XX |
| 3135-6 | — | — | — | — | — | — | — | — | YY | — | — | — |
| 3106-4 | — | YY | — | XX | — | XX | — | YY | XX | XX | YY | — |
| 3106-2 | — | — | — | — | — | — | XX | — | — | — | — | — |
| 3106-7 | YY | — | — | XX | XX | — | YY | XX | XX | YY | YY | — |

TABLE 5
PANEL 11 RUN WITH COMPROMISED NUCLEIC ACID SAMPLES SET C. THE SOURCES
OF THE COMPROMISED SAMPLES OF SET C ARE DESCRIBED IN TABLE 8.

| | 212605 | 220875 | 65882 | 57575 | 66683 | 214674 | 248007 | 63804 | 56144 | 233357 | 60188 | 206538 |
|---|---|---|---|---|---|---|---|---|---|---|---|---|
| 3260-1 | XY | — | — | — | — | — | — | XX | — | — | XX | XX |
| 3135-4 | — | — | — | — | — | YY | YY | YY | — | — | YY | YY |
| 3135-5 | — | — | — | — | — | — | — | — | — | — | — | — |
| 3135-6 | YY | — | — | — | — | — | — | — | — | — | — | — |
| 3106-4 | — | — | — | — | XX | — | YY | XX | XX | YY | — | — |
| 3106-2 | YY | — | — | — | XX | — | — | — | — | — | — | — |
| 3106-7 | — | — | XY | XY | — | — | — | — | YY | — | YY | YY |

TABLE 6
PANEL 9 RUN WITH COMPROMISED NUCLEIC ACID SAMPLES SET C.
THE SOURCES OF THE COMPROMISED SAMPLES OF SET C ARE
DESCRIBED IN TABLE 8.

| | 56593 | 217856 | 231735 | 81917 | 62684 | 241554 | 126264 | 224922 | 81081 | 66561 | 63799 | 119770 |
|---|---|---|---|---|---|---|---|---|---|---|---|---|
| 3260-1 | — | — | — | — | — | — | — | — | — | — | — | — |
| 3135-4 | — | XY | YY | XX | — | YY | XX | — | — | YY | — | XX |
| 3135-5 | XY | — | — | — | — | — | — | — | — | — | XX | XX |
| 3135-6 | — | — | — | — | — | — | — | — | — | — | — | — |
| 3106-4 | YY | — | — | — | XX | — | XX | — | YY | — | — | YY |
| 3106-2 | XX | — | XX | — | — | — | — | — | XX | — | — | XX |
| 3106-7 | — | YY | — | — | — | XX | — | — | — | — | YY | — |

TABLE 7
PANEL 10 RUN WITH COMPROMISED NUCLEIC ACID SAMPLES SET C. THE SOURCES OF THE
COMPROMISED SAMPLES OF SET C ARE DESCRIBED IN TABLE 8.

| | 63836 | 60676 | 58091 | 68909 | 238155 | 201688 | 57849 | 56915 | 56608 | 68532 | 61500 | 66026 |
|---|---|---|---|---|---|---|---|---|---|---|---|---|
| 3260-1 | — | — | — | — | — | — | — | — | — | — | — | — |
| 3135-4 | YY | — | XX | YY | XY | — | XY | XY | YY | XX | — | YY |
| 3135-5 | — | — | — | — | — | — | — | — | — | — | — | — |
| 3135-6 | — | — | — | — | — | — | — | — | — | — | — | — |
| 3106-4 | YY | —Y | XX | YY | — | XX | XX | XX | — | XX | YY | XX |
| 3106-2 | XY | — | — | — | — | — | — | — | — | — | — | X— |
| 3106-7 | — | — | XX | XX | — | — | XX | — | — | — | — | XX |

TABLE 8
SOURCES OF THE COMPROMISED NUCLEIC
ACID SAMPLES OF SET C.

| SAMPLE | | # OF SNPs | # OF SNPs |
|---|---|---|---|
| NUMBER | SAMPLE TYPE | ATTEMPTED | SUCCESSFUL |
| 3260-1 | Bone, found on a riverbank | 60 | 4 |
| 3135-4 | Hair (bleached) taken from | 60 | 35 |
| | under a vehicle | | |
| 3135-5 | Hair (bleached) taken from | 60 | 7 |
| | under a vehicle | | |
| 3135-6 | Hair (bleached) taken from | 60 | 2 |
| | under a vehicle | | |
| 3106-4 | Hair (reference) | 60 | 31 |
| 3106-2 | Possible hair (African | 60 | 10 |
| | American) from vacuum | | |
| | sweeping | | |
| 3106-7 | Swab from a necklace | 60 | 25 |

TABLE 9
RESULTS OF COMPROMISED DNA SAMPLES

| | | | # OF SNPs | # OF SNPs | FREQ. OF SNP |
|---|---|---|---|---|---|
| SAMPLE # | AMEL. | # OF STRs | ATTEMPTED | SUCCESSFUL | PROFILE |
| 231965 | XY | 3 | 60 | 12 | 13,908 |
| 12792 | X | 1 | 48 | 31 | 1.22 × 10⁹ |
| 12793 | XY | 1 | 24 | 17 | 37,594 |
| 14704 | X | 5 | 24 | 17 | N.D. |
| 14686 | XY | 3 | 24 | 18 | N.D. |
| 231749 | XY | 1 | 60 | 45 | 1.4 × 10¹⁰ |
| 14700 | XY | 5 | 60 | 56 | N.D. |
| 14775 | XX | 4 | 60 | 57 | N.D. |
| 232121 | XX | 6 | 60 | 59 | N.D. |
| 231770 | XY | 2 | 60 | 60 | 1.78 × 10²² |

TABLE 10
COMPROMISED NUCLEIC ACIDS ANALYZED BY PANELS IN
ACCORDANCE WITH THE PRESENT INVENTION.

| | | | # OF SNP | # OF SNP |
|---|---|---|---|---|
| SAMPLE # | PROT+ | COF | ATTEMPTED | SUCCESSFUL |
| 524-3A | 1 locus | XY only | 71 | 63 |
| 590-2A | 3 loci | XY only | 71 | 68 |
| 617-1A | XY only | XY only | 71 | 53 |
| 660-1A | XY only | NEG | 71 | 55 |
| 667-1A | NEG | XY only | 71 | 64 |
| 1268-1A | NEG | XY only | 71 | 65 |
| 1300-1A | NEG | NEG | 71 | 16 |
| 1337-2A | 4 loci | 2 loci | 71 | 70 |
| 1233-1A | NEG | NEG | 71 | 65 |
| 1473-2A | 4 loci | 1 locus | 71 | 68 |
| 1476-1A | NEG | 1 locus | 71 | 63 |
| 1477-1A | 2 loci | 3 loci | 71 | 59 |
| 1462-1A | 1 locus | NEG | 71 | 63 |
| 1514-1A | NEG | NEG | 71 | 47 |
| 1526-1A | NEG | XY only | 71 | 57 |
| 1650-2A | 4 loci | 2 loci | 71 | 68 |
| 1747-1A | 1 locus | NEG | 71 | 67 |
| 1818-1A | 4 loci | 2 loci | 71 | 68 |
| 1819-1A | XY only | XY only | 71 | 71 |
| 1945-1A | 1 locus | NEG | 71 | 50 |
| 1946-1A | 4 loci | 2 loci | 71 | 63 |
| 2163-1B | 6 loci | 4 loci | 71 | 68 |
| 2181-1B | NEG | NEG | 71 | 62 |

TABLE 11
SUMMARY OF 44 PERSON STUDY -
24,640 POSSIBLE GENOTYPES

| | | | NO. OF | |
|---|---|---|---|---|
| AMOUNT OF | 70 SNPs NO. | % OF | INCORRECT | % OF |
| DNA | OF FL | TOTAL | TYPINGS | TOTAL |
| 2 ng | 81 | 97.37 | 0 | 100 |
| 320 pg | 159 | 94.84 | 4 | 99.86 |
| 240 pg | 145 | 95.29 | 9 | 99.69 |
| 160 pg | 75 | 97.56 | 9 | 99.7 |
| 80 pg | 140 | 95.45 | 12 | 99.55 |
| 40 pg | 223 | 92.76 | 63 | 97.79 |
| 20 pg | 458 | 85.13 | 146 | 94.43 |
| 10 pg | 1090 | 64.64 | 220 | 88.95 |
| | 2370 | 90.38 | 463 | 97.92 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. A panel of single nucleotide polymorphisms for analyzing compromised nucleic acid samples, comprising two or more single nucleotide polymorphisms, wherein each of the two or more single nucleotide polymorphisms of the panel are selected from single nucleotide polymorphisms that are not genetically linked with respect to one another, and wherein each of the two or more single nucleotide polymorphisms of the panel are selected from single nucleotide polymorphisms that are located outside tandem repeat nucleic acid sequences.

2. A panel according to claim 1, wherein the single nucleotide polymorphisms include the nucleic acid sequences selected from the group consisting of SEQ ID NOS. 25-36, 61-72, 98-109, 134-145, 170-181, 206-217, 242-253, 278-289, 314-325, 351-362, 387-398, 423-434, and 457-467.

3. A method of generating a panel of single nucleotide polymorphisms from a population of interest for analyzing a compromised nucleic acid sample, comprising:
selecting a panel of two or more single nucleotide polymorphisms in a genome of the population of interest, wherein each of the two or more single nucleotide polymorphisms of the panel are single nucleotide polymorphisms of the genome that are not genetically linked with respect to one another, and wherein each of the two or more single nucleotide polymorphisms of the panel are single nucleotide polymorphisms of the genome that are located outside tandem repeat nucleic acid sequences, thereby generating the panel of single nucleotide polymorphisms from the population of interest for analyzing the compromised nucleic acid sample.

4. A method according to claim 3, wherein the compromised sample comprises nucleic acids from about 10 nucleotides in length to about 100 nucleotides in length.

5. A method according to claim 3, wherein the population of interest is human.

6. A method according to claim 3, wherein the population of interest is one missing human.

7. A method for determining the identity of an individual from an unknown sample of compromised nucleic acids, comprising:
obtaining the unknown sample of compromised nucleic acids having two or more single nucleotide polymorphisms from an individual;
identifying two or more single nucleotide polymorphisms present in the unknown sample of compromised nucleic acids;
comparing the identity of each of the two or more single nucleotides polymorphisms in the compromised sample with a panel of single nucleotide polymorphisms from a known sample to determine a number of matches between each of the two or more single nucleotide polymorphisms in the unknown sample and the panel, wherein the panel comprises two or more single nucleotide polymorphisms that are not genetically linked with respect to one another, and are located outside tandem repeat nucleic acid sequences; and
determining the probability that the unknown sample and the known sample are derived from the same or related individual based on the number of matches between each of the two or more single nucleotide polymorphism in the unknown sample and the known sample, thereby determining the identity of the individual from the unknown sample of compromised nucleic acids.

8. A method for determining the identity of an individual from an unknown sample of compromised nucleic acids, comprising:
obtaining the unknown sample of compromised nucleic acids having two or more single nucleotide polymorphisms from an individual;
obtaining a known sample of nucleic acids having two or more single nucleotide polymorphisms;
selecting a panel of two or more single nucleotide polymorphisms, wherein each of the two or more single nucleotide polymorphisms of the panel are not genetically linked with respect to one another, and wherein each of the single nucleotide polymorphisms of the panel are located outside tandem repeat nucleic acid sequences;
determining the identity of each of the two or more single nucleotide polymorphisms of the panel that are present in the compromised nucleic acid sample; and
determining the identity of each of the two or more single nucleotide polymorphisms of the panel that are present in the known sample;
comparing the identities of the two or more single nucleotide polymorphisms of the panel observed in the known sample with the identities of the two or more single nucleotide polymorphisms of the panel observed in the unknown sample of compromised nucleic acids; and
determining the probability that the unknown sample and the known sample are derived from the same or related individual, thereby determining the identity of the individual from the unknown sample of compromised nucleic acids.

9. A method according to claim 7, wherein the known sample and the unknown sample are from the same individual.

10. A method according to claim 7, wherein the known sample is from a family member.

11. A method according to claim 7, wherein the compromised nucleic acid sample comprises nucleic acid fragments from about 10 nucleotides in length to about 100 nucleotides in length.

12. A method according to claim 7, wherein the identity of the one or more single nucleotide polymorphisms is determined using a single base primer extension reaction.

13. A method according to claim 7, wherein the two or more of the single nucleotide polymorphisms of the compromised sample are identified in a multiplexed reaction.

14. A method according to claim 7, wherein the two or more of the single nucleotide polymorphisms of the panel are identified in a multiplexed reaction.

15. A method according to claim 7, wherein the two or more single nucleotide polymorphisms of the panel are identified on an array.

16. A method according to claim 7, wherein the two or more single nucleotide polymorphisms of the compromised sample are identified on an array.

17. A method according to claim 15, wherein the array is an addressable array.

18. A method according to claim 16, wherein the array is an addressable array.

19. A method according to claim 15, wherein the array is a virtual array.

20. A method according to claim 16, wherein the array is a virtual array.

21. A method for genotyping a compromised nucleic acid sample, comprising
obtaining the sample of compromised nucleic acids from an individual;
identifying two or more single nucleotide polymorphisms present in the compromised nucleic acid sample; and
comparing the identity of each of the two or more single nucleotides polymorphisms in the compromised sample with a panel of single nucleotide polymorphisms from a population of interest to determine the frequency of occurrence of each of the two or more single nucleotide polymorphism in the compromised sample with the population of interest, wherein the panel comprises two or more single nucleotide polymorphisms that are not genetically linked with respect to one another, and are located outside tandem repeat nucleic acid sequences; thereby genotyping the sample of compromised nucleic acids.

22. A method for genotyping a compromised nucleic acid sample, comprising
obtaining the sample of compromised nucleic acids from an individual;
selecting a panel of single nucleotide polymorphisms from a genome of a population of interest, the panel comprising two or more single nucleotide polymorphisms, wherein each of the two or more single nucleotide polymorphisms of the panel are single nucleotide polymorphisms that are not genetically linked with respect to one another and are located outside tandem repeat nucleic acid sequences;
identifying two or more single nucleotide polymorphisms present in the compromised nucleic acid sample; and
comparing the identities of the two or more single nucleotide polymorphisms observed in the compromised sample with the identities of the two or more single nucleotide polymorphisms observed in the panel to determine a genotype, thereby obtaining the genotype for the compromised nucleic acid sample.

23. A genotyping method according to claim 22, wherein the single nucleotide polymorphisms are biallelic and the identities of the alleles of the single nucleotide polymorphisms are T and/or C.

24. A genotyping method according to claim 22, wherein the population of interest is human.

25. A genotyping method according to claim 22, wherein the sample comprises human nucleic acids.

26. A genotyping method according to claim 22, wherein the two or more single nucleotide polymorphisms present in the compromised nucleic acid sample are identified using a single base primer extension reaction.

27. A genotyping method according to claim 22, wherein the two or more single nucleotide polymorphisms present in the compromised nucleic acid sample are identified in a multiplexed reaction.

28. A genotyping method according to claim 22, wherein the two or more single nucleotide polymorphisms present in the compromised nucleic acid sample are identified on an array.

29. A genotyping method according to claim 28, wherein the array is an addressable array.

30. A genotyping method according to claim 28, wherein the array is a virtual array.

31. A genotyping method according to claim 22, wherein the compromised nucleic acid sample is amplified to a length of from about 10 nucleotides to about 100 nucleotides.
